(19) United States
(12) Patent Application Publication
(10) Pub. No.: US 2025/0027168 A1
(43) Pub. Date: Jan. 23, 2025

(54) DETECTING DISEASE-ASSOCIATED TARGET NUCLEIC ACIDS IN A MAMMAL AND TREATMENT THEREOF

(71) Applicants: The Regents of the University of California, La Jolla, CA (US); South Australian Health & Medical Research Institute, Adelaide (AU)

(72) Inventors: Robert Cooper, San Diego, CA (US); Jeff Hasty, Encinitas, CA (US); Daniel Lindsay Worthley, Adelaide (AU); Susan Lesley Woods, Adelaide (AU); Josephine Wright, Adelaide (AU)

(21) Appl. No.: 18/778,195

(22) Filed: Jul. 19, 2024

Related U.S. Application Data
(60) Provisional application No. 63/528,234, filed on Jul. 21, 2023.

(51) Int. Cl. C12Q 1/6886; C12N 9/22; C12N 15/11; C12N 15/90; C12Q 1/02; C12Q 1/44; C12Q 1/6888

(52) U.S. Cl. CPC C12Q 1/6886; C12N 9/22; C12N 15/111; C12N 15/902; C12Q 1/02; C12Q 1/44; C12Q 1/6888; C12N 2310/20; G01N 2333/922

(57) ABSTRACT

Disclosed herein are methods of detecting a target nucleic acid in a sample (e.g., biological sample or environmental sample). The method includes administering to the sample an effective amount of a composition comprising an engineered bacterium (e.g., A. baylyi) and detecting an output signal. In some cases, the mammal has a cancer or an infectious disease.

### CLAIM OF PRIORITY

This application claims the benefit of U.S. Provisional Application Ser. No. 63/528,234, filed on Jul. 21, 2023. The entire contents of the foregoing are incorporated herein by reference.

### FEDERALLY SPONSORED RESEARCH OR DEVELOPMENT

This invention was made with government support under GM069811 and CA241728, awarded by the National Institutes of Health. The government has certain rights in the invention.

### SEQUENCE LISTING

This application contains a Sequence Listing that has been submitted electronically as an XML file named “15670-0375001 ST26 SL.XML.” The XML file, created on Jul. 12, 2024, is 9,887 bytes in size. The material in the XML file is hereby incorporated by reference in its entirety.

### TECHNICAL FIELD

The present disclosure relates to detecting a target nucleic acid (e.g., colorectal cancer (CRC) DNA) in a sample using biosensing bacteria (e.g., genetically engineered bacteria).

### BACKGROUND

Natural competence is one mechanism of horizontal gene transfer (HGT), the exchange of genetic material between organisms outside vertical, “parent to offspring,” transmission (12). HGT is common between microbes (Soucy et al., Nature Reviews Genetics. 16, 472-482 (2015)). It can also occur from microbes into animals and plants (Robinson et al., Plos Genet. 9, e1003877 (2013)) and, in the opposite direction, from eukaryotes to prokaryotes (Hotopp. Trends Genet. 27, 157-163 (2011)). However, forward engineering of bacteria to detect and respond to mammalian DNA via HGT has not been explored. Many existing techniques for detecting and analyzing DNA require extensive processing steps to extract and purify samples, and most require expensive equipment and personnel with significant training and expertise. Thus, there is an unmet clinical need for low-cost, low-labor, and equipment-free detection of target nucleic acid (e.g., DNA).

### SUMMARY

Provided herein are methods of detecting a target nucleic acid in a sample, the method including: (a) administering to the sample an effective amount of a composition including a bacterium, where the bacterium includes: a competence system including a homologous recombination construct, where a first homology arm is substantially complementary to a sequence upstream of the target nucleic acid and a second homology arm is substantially complementary to a sequence downstream of the target nucleic acid; an output gene construct encoding an output signal, and optionally; a CRISPR/Cas system including an effector nuclease and a spacer directed to a non-targeted nucleic acid; and (b) detecting the output signal.

In some embodiments, the output signal is repressed by a repressor. In some embodiments, the repressor is located between the two homology arms. In some embodiments, the output signal is repressed by DNA looping with a repressor binding site located outside a CRISPR-targeted transposon that recognizes the target nucleic acid. In some embodiments, the methods provided herein includes a nuclease inactivated CRISPR effector fused to a portion of a split enzyme, where the CRISPR effector recognizes the target nucleic acid in the sample to produce the output signal of an activated split enzyme. In some embodiments, the repressor is a polypeptide repressor, an RNA-based repressor, or a CRISPR Cas system. In some embodiments, the polypeptide repressor includes a tetracycline repressor (tetR),a lac repressor (lacI), a CI repressor, a PhIF repressor, a IcaRA repressor, an AmtR repressor, a BetI repressor, a SrpR repressor, an Orf2 repressor, a BM3RI repressor, a ButR repressor, a PhIF repressor, an AmeR repressor, a QacR repressor, a LmrA repressor, a PsrA repressor, a HlyIIR repressor, a McbR repressor, a ScbR repressor, a TarA repressor, a LitR repressor, a HapR repressor, or a SmcR repressor. In some embodiments, the RNA-based repressor includes a small RNA-based repressor, toehold repressor, 3-way junction repressor, pT181 dual transcriptional/translational repressor. In some embodiments, the CRISPR Cas system includes CRISPR inactivation system.

In some embodiments, the sample is an environmental sample or a biological sample. In some embodiments, the environmental sample includes an air sample, a water sample, a dust sample, an agricultural product sample, a plant sample, a sewage sample, or a soil sample. In some embodiments, the biological sample is from an animal.

In some embodiments, competence system includes a type IV pili. In some embodiments, the target nucleic acid includes a portion of an oncogene, a bacterial gene, or a viral gene.

In some embodiments, the CRISPR/Cas system includes an I-A, I-B, I-C, I-D, I-E, I-F1, I-F2, I-F3, I-G, II-A, II-B, II-C1, II-C2, III-A, III-B-, III-C, III-D, III-E, III-F, IV-A, IV-B, IV-C, V-A, V-B1, V-B2, V-C, V-D, V-E, V-F1, V-F2, V-F3, V-G, V-U1, V-U2, V-U4, V-U5, VI-A, VI-B1, VI-B2, VI-C, or a VI-D CRISPR-Cas system.

In some embodiments, the bacterium includes Acinetobacter baylyi, Bacillus subtilis, Streptococcus pneumoniae, Neisseria gonorrhoeae, Haemophilus influenzae, Vibrio natriegens, Vibrio cholera or Streptococcus sanguinis.

In some embodiments, the oncogene is Kirsten rat sarcoma virus (KRAS), Tumor protein p53 (TP53), Mothers against decapentaplegic homolog 4 (SMAD4), or V-Raf murine sarcoma viral oncogene homolog B1 (BRAF). In some embodiments, the animal is a mammal having a cancer or an infectious disease. In some embodiments, the cancer is colorectal cancer (CRC) or pancreatic cancer. In some embodiments, the infectious disease is a microbial infection or an antimicrobial resistant infection.

In some embodiments, the cancer is characterized by a mutated KRAS gene, a mutated TP53, a mutated SMAD4, or a mutated BRAF gene. In some embodiments, the mutated KRAS gene includes a G12D, a G13, or a Q61 mutation. In some embodiments, the mutated TP53 gene includes a R175 or a R248 mutation. In some embodiments, the mutated SMAD4 gene includes a R361 or a R265 mutation. In some embodiments, the mutated BRAF gene includes a BRAFV600E mutation.

In some embodiments, the output signal includes an antibiotic resistance polypeptide, a fluorescent polypeptide, a polypeptide which reacts with a substrate to produce luminescence, a polypeptide that causes a color change, an ultrasound contrast agent polypeptide, a lateral flow assay, or an activated split enzyme.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Engineered bacteria to detect tumor DNA. Engineered Acinetobacter baylyi bacteria were delivered rectally in an orthotopic mouse model of CRC. The naturally competent A. baylyi take up tumor DNA shed into the colorectal lumen. The tumor donor DNA is engineered with a kanR cassette flanked by KRAS homology arms. The sensor bacteria were engineered with matching KRAS homology arms that promote homologous recombination. Sensor bacteria that undergo HGT from tumor DNA acquire kanamycin resistance and were quantified from luminal contents by serial dilution on antibiotic selection plates.

FIGS. 2A-2J. Sensing KRASG12D DNA in vitro. FIG. 2A: Donor DNA derived from plasmid, purified cancer cell genomic DNA, or raw lysate (top) was recombined into biosensor A. baylyi cells (bottom). Horizontal gene transfer included either a large, 2-kb insert (FIG. 2B) or a small, 8-bp insert to repair two stop codons (FIG. 2C), in both cases conferring kanamycin resistance. FIGS. 2D-2G: A. baylyi biosensors were incubated with plasmid DNA, purified RKO-KRAS or LS174T-KRAS genomic DNA, or raw RKO-KRAS lysate, all containing the donor cassette, or purified RKO or LS174T genomic DNA as controls. Biosensor cells included either “large insert” (FIG. 2B, FIG. 2D, and FIG. 2E) or “small insert” (FIG. 2C, FIG. 2F, and FIG. 2G) designs, and transformations were performed in liquid culture (FIG. 2D and (FIG. 2F) or on solid agar surfaces (FIG. 2E and FIG. 2G). Two sample t tests was used to compare the data of RKO and LS174T genomic DNA controls under the same conditions. FIG. 2H: CRISPR spacers targeting the KRAS G12D mutation (boxed), using the underlined PAMs. FIG. 2I and FIG. 2J: Fraction of total biosensor cells expressing the indicated CRISPR spacers that were transformed by plasmid donor DNA with wild type (FIG. 2I) or mutant G12D (FIG. 2J) KRAS. Statistics were obtained by using two sample, one-sided/tests, with P values displayed. Data points below detection are shown along the x axis, at the limit of detection.

FIGS. 3A-3G. Detection of donor DNA from BTRZI-KRAS-kanR organoids in both in vitro and in vivo models of colorectal cancer. FIG. 3A: Schema depicting in vitro coculture of A. baylyi sensor bacteria with BTRZI-KRAS-kanR (CRC donor) organoid lysates or viable organoids to assess HGT repair of kanamycin resistance gene (kanR). FIG. 3B: Recombination with DNA from crude lysates enables growth of A. baylyi sensor on kanamycin. FIG. 3C: Representative images of GFP (Green Fluorescent Protein)-tagged A. baylyi biosensor surrounding parental BTRZI (control) and BTRZI-KRAS-kanR donor organoids at 24 hours. Scale bar, 100 mm. FIG. 3D: Coculture of established CRC BTRZI-KRAS-kanR donor organoids with A. baylyi sensor enables growth of A. baylyi sensor on kanamycin. In (FIG. 3B) and (FIG. 3D), n=5 independent experiments each with 5 technical replicates; one sample t test on transformed data was used for statistical analysis with P values as indicated. FIG. 3E: Schema depicting in vivo HGT experiments: generation of BTRZI-KRAS-kanR (CRC donor) tumors in mice through colonoscopic injection, with tumor pathology validated by hematoxylin and eosin (H&E) histology, administration of biosensors, and analysis of luminal contents. Scale bars, 200 mm. FIG. 3F: Rectal delivery of A. baylyi biosensor to mice bearing CRC donor tumors results in kanamycin-resistant A. baylyi biosensor in luminal contents through HGT with transformation efficiency of 1.5×10⁻⁹ (limit of detection 1.25×10⁻¹⁰). HGT rate calculated from colony-forming units (CFU) on kanamycin-chloramphenicol-vancomycin (transformants) and chloramphenicol-vancomycin (total A. baylyi) selection plates, n=3 to 5 mice per group. One-way analysis of variance with Tukey's post-hoc on log₁₀-transformed data was used for statistical analysis. FIG. 3G: ROC (receiver operating characteristic) curve analysis of HGT CFU following enema; area under the curve=1, P=0.009.

FIGS. 4A-4E. Detection of nonengineered DNA. FIG. 4A: tetR located between the homology arms on the A. baylyi genome represses expression of the output gene. FIG. 4B: Target DNA with wild-type KRAS sequence is recognized and degraded by the type I-F CRISPR-Cas effector complex, Cascade. FIG. 4C: Target DNA with the KRASG12D mutation avoids degradation, replaces tetR in the biosensor genome, and relieves repression of the output gene. (FIG. 4D and FIG. 4E) Fraction of biosensors with either a random CRISPR spacer (FIG. 4D) or a spacer targeting wild-type KRAS (FIG. 4E) that detected donor DNA. Statistics were obtained in two sample t tests.

FIG. 5. Engineered donor construct. Plasmid donor DNA used to transfect mammalian cell lines and used as positive control donor DNA for in vitro experiments.

FIGS. 6A-6B. Engineered landing pad within the biosensor to receive target DNA. “Large insert” (FIG. 6A), and “small insert” (FIG. 6B), designs for the biosensors. KRAS homology arms are shown in striped, gray with surrounding genomic context outside them. Note that large and small inserts refer to the size of the donor DNA region that must transfer to confer kanamycin resistance, not to the size of the region between homology arms in the biosensor. Two single-base changes introducing nearby stop codons at the beginning of kanR are shown for the small insert design, (FIG. 6B).

FIGS. 7A-7C. Copy number of target DNA within cancer cell and organoid lines. FIG. 7A: On average, 1 to 3 copies of target donor DNA were integrated per cell into polyclonal donor cell and organoid lines. The number of copies of target donor DNA integrated per cell was quantitated using qPCR with primers designed to amplify the KRAS homology arms in the donor cassette, as well as endogenous human KRAS. Parental RKO and LS174T cell lines were diploid for human KRAS (2n), whereas human KRAS could not be amplified from parental BTRZI mouse organoid genomic DNA. Donor cells had endogenous KRAS locus (2n) as well as 2.5±0.6 (RKO) and 1.4±0.2 (LS174T) copies of target donor DNA. BTRZI donor organoids had 2.0±0.8 copies of target donor DNA (mouse endogenous KRAS locus not detectable by human specific primers). n=3 independent gDNA extractions per sample with each qPCR were conducted in triplicate and compared to a standard curve generated with normal human gDNA. FIG. 7B: LS174T cells were transduced with different multiplicity of infection (MOI) and stable cell lines were selected with 2 μg/ml puromycin. Donor cells had endogenous KRAS locus (2n) and 1.4 (MOI 0.1), 2.4 (MOI 0.7) and 4.7 (MOI 3.5) copies of target donor DNA. FIG. 7C: Recombination with donor DNA from crude lysates of LS174T donor cell lines with different numbers of target donor DNA integrated per cell enabled the growth of A. baylyi sensor on kanamycin plates. n=5 independent experiments each with 5 technical replicates. Paired t-test showed that there was no significant difference in the recombination efficiency.

FIGS. 8A-8D. Detection of donor DNA from both microsatellite stable (MSS) and microsatellite instable (MSI) colorectal cell lines and human organoids. FIG. 8A: Images of human CRC cell lines, SW620 (MSS) and LS174T (MSI-H), with the donor cassette stably integrated into their genome. Scale bar 200 μm. FIG. 8B: Recombination with DNA from crude lysates of human CRC donor cell lines enabled growth of A. baylyi sensor on kanamycin plates.

FIG. 8C: Images of human CRC organoids established from patient surgical samples with the donor cassette stably integrated into their genome. Scale bar 200 μm. FIG. 8D: Recombination with DNA from crude lysates of human CRC donor organoid lines enabled growth of A. baylyi sensor on kanamycin plates. FIG. 8B and FIG. 8D: n=5 independent experiments each with 5 technical replicates. Paired t-test showed that there was no significant difference in recombination efficiency between MSS and MSI patient organoids.

FIGS. 9A-9C. Sensor detection of donor DNA from BTRZI CRC organoids. A. baylyi sensor bacteria were constitutively chloramphenicol-resistant, hence chlorR CFUs provided a readout of total A. baylyi present. In contrast, kanamycin-resistant sensor bacteria relied on incorporation of donor DNA from CRC organoids to correct the defective kanR gene and enable growth on kanamycin selection plates. FIG. 9A: Recombination with lysate from CRC donor organoids enabled growth of A. baylyi sensor on kanamycin plates. Shown here are the representative plates and CFU analysis. FIG. 9B: After co-culturing established CRC donor organoids with A. baylyi sensor, recombination with donor DNA from CRC donor organoids enabled growth of A. baylyi sensor on kanamycin plates. Shown are representative images and CFU analysis. Scale bars 200 μm. FIG. 9A and FIG. 9B: FIG. 3 contains the same data as shown here but presented as HGT rate (kanamycin resistant CFU A. baylyi per mL/chloramphenicol CFU A. baylyi per mL), n=5 independent experiments each with 5 technical replicates. FIG. 9C: Representative Sanger sequencing chromatograms of PCR amplicon covering the region of the kanR gene containing informative SNPs are shown to highlight the difference in sequence of gDNA isolated from the parental A. baylyi sensor bacteria compared to the A. baylyi colonies isolated from kanamycin plates following mixing with the donor organoid lysates or viable organoids.

FIG. 10. Detection of target DNA mixed with stool. A. baylyi sensor bacteria were mixed with serially diluted donor plasmid DNA in the presence of mouse stool collected from mice bearing CRC tumors (1 stool per reaction). After 3 hours of growth at 37° C., serial dilutions of each reaction were plated onto kanamycin/vancomycin LB agar selection plates (A. baylyi sensor HGT) or chloramphenicol/vancomycin LB agar selection plates (total A. baylyi sensor). Rate of HGT was calculated (p-values calculated on log-transformed data using unpaired t-test). n=5 independent experiments, each with 5 technical replicates.

FIGS. 11A-11B. High sensitivity digital droplet PCR (ddPCR) detection of CRC mutation (BrafV600E) in stool DNA was isolated from tumor-bearing animals. FIG. 11A: Representative images of ddPCR data. FIG. 11B: CRC mutation (BrafV600E) positive droplets as a % of total droplets. Analysis of no template negative control samples and stool DNA samples from non-tumor bearing animals were used to determine the sensitivity threshold of the assay. +ve ctrl=positive control samples that contained 10% BrafV600E gDNA spiked into the stool DNA sample from a non-tumor bearing animal, NT=no tumor, Ts=small tumor, Tm=medium tumor, Tl=large tumor, NTC=no template PCR negative control (n=3-4 mice/group).

FIGS. 12A-12C. Horizontal gene transfer was detected in luminal contents from mice bearing BTRZI CRC donor tumors after rectal dosing of A. baylyi sensor bacteria. FIGS. 12A-12B: Recombined transformants were GFP positive and confirmed as A. baylyi by 16S sequencing on kanamycin/chloramphenicol/vancomycin selection plates; scale bar 500 μm. FIG. 12C: Representative Sanger sequencing chromatograms of PCR amplicon covering the region of the kanR gene containing informative SNPs are shown to highlight the difference in sequencing DNA isolated from the parental A. baylyi sensor bacteria compared to the A. baylyi colonies isolated from kanamycin/chloramphenicol/vancomycin plates in luminal contents from mice bearing BTRZI CRC donor tumors after rectal dosing of A. baylyi sensor bacteria.

FIGS. 13A-13B. Schematics of a lateral flow assay. FIG. 13A: Custom lateral flow strips contain immobilized anti-6×His at the control line and 150 nm gold nanoshells were conjugated to anti-FLAG isolated from mouse at the conjugate pad. Samples containing secreted Hcp-6×His-FLAG is applied to the sample pad, from which it is wicked rightward via capillary action. Sample is first interacted with anti-FLAG-nanogold at the conjugate pad, and then binds to anti-6×His at the test line. Remaining gold nanoparticles bind to the test line. FIG. 13B: Detection of supernatant from 10⁷ (top) or 10⁶ (middle) engineered A. baylyi, or from the wild-type as a negative control (bottom). Control line left, test line right.

FIGS. 14A-14C. Schematics of CRISPR-associated transposon biosensors. FIG. 14A: The output gene is embedded inside a site-specific, CRISPR-targeted transposon (end sequence as red triangles). DNA looping between LacI, weakly bound to the promoter-adjacent lacO3 operator, and strongly bound to the lacO1 operator outside the transposon represses gene expression. FIG. 14B: DNA-of-interest containing a target for the transposon recombines into a second site on the genome. FIG. 14C: The transposon jumps to its target site, eliminating the DNA looping and allowing nearly full output expression, with the weak lacO3 operator only poorly binding the LacI on its own.

FIG. 15. DNA uptake in human samples. A. baylyi were incubated with donor DNA in LB mixed with human saliva and urine samples at 0-50% of total volume. The fraction of cells taking up the donor DNA were quantified by serial dilution on selection plates (left panel). A. baylyi colony forming units (CFUs) in the urine and saliva samples on selection plates were quantified.

### DETAILED DESCRIPTION

In vitro DNA analysis helps detect and manage many human diseases, including cancer and infection (30). However, in vitro sensing requires potentially invasive removal of samples, and many DNA diagnostics do not achieve clinically relevant sequence resolution, with more advanced techniques remaining too expensive for routine use in many settings (31). In addition to well-informed treatment plans for individual patients, an effective public health system also relies on diagnostic tests. Culture tests remain a widespread standard for diagnosing infectious diseases, but such tests take long to provide point-of-care guidance for treatment, and do not identify all microbes. Nearly all diagnostic technologies demand capital equipment that must be maintained and supplied with electricity, and they involve multiple manual steps to isolate cultures and purify DNA. Two of the most affordable and easy-to-use technologies are immunological assays, and paper-based in vitro CRISPR assays. However, these technologies still require sample pre-processing (DNA isolation and amplification). Centers for Disease Control and Prevention (CDC) identified increasingly prevalent low-resolution, culture-independent diagnostic tests as a growing threat to public health monitory. Thus, point-of-care diagnostic strategies and tests that are modular and easy to adapt to new targets remain an unmet clinical need.

Provided herein are living biosensors (e.g., engineered bacteria) that can be engineered to detect target nucleic acid (e.g., cell-free DNA) sequences or mutations, and methods of use thereof. For example, forward engineering of bacteria (e.g., Acinetobacter baylyi) can detect and respond to nucleic acids (e.g., DNA or RNA) through horizontal gene transfer (e.g., natural competence). Also included is the in vitro characterization of functionalities of the biosensors using coculture assays, followed by in vivo validation in mice. Also provided herein are methods and materials for detection of target nucleic acid in a sample (e.g., biological sample or environmental sample). For example, the biosensor can be engineered to detect both engineered as well as natural or nonengineered tumor DNA sequences (e.g., KRASG12D). Furthermore, the biosensor can be adapted to detect arbitrary nucleic acid sequences (e.g., oncogenes, bacterial gene, or viral gene) without requiring sample pre-processing (e.g., DNA purification or RNA purification), thus enabling a more affordable and rapid detection of various diseases (e.g., cancer or infectious diseases).

### Living Biosensors

Living synthetic bacteria have many advantages for biosensing applications, including their ability to detect signals in situ, capacity for information processing, potential case-of-use, and low cost. Bacterial engineering has allowed the development of living cell diagnostics and therapeutics (1-3). Some bacteria are naturally competent for transformation and can sample extracellular DNA directly from their environment (11). For example, Acinetobacter baylyi (A. baylyi) is a highly competent bacterium (15, 16) that is largely nonpathogenic in healthy humans (17). A. baylyi has been shown to colonize murine gastrointestinal tract (18) and acquires unpurified environmental DNA from the lysed cells (19). A. baylyi is competent throughout its life cycle and does not rely on any particular signal sequence to initiate DNA uptake.

In vitro CRISPR-Cas technology has been used as biosensors, but its fails to provide case of use, minimization of sample preparation, and elimination of equipment that requires capital expenditure and maintenance. Sample preparation is particularly difficult to avoid for in vitro use, since enzymes require precise buffer conditions for optimal activity. Therefore, nucleic acids (e.g., DNA or RNA) in complex biological samples are generally centrifuged to remove debris, extracted, and either eluted into another buffer or greatly diluted. Another challenge for in vitro CRISPR-Cas biosensors is achieving high sensitivity without additional amplification steps since each activated enzyme can produce only a limited amount of signal.

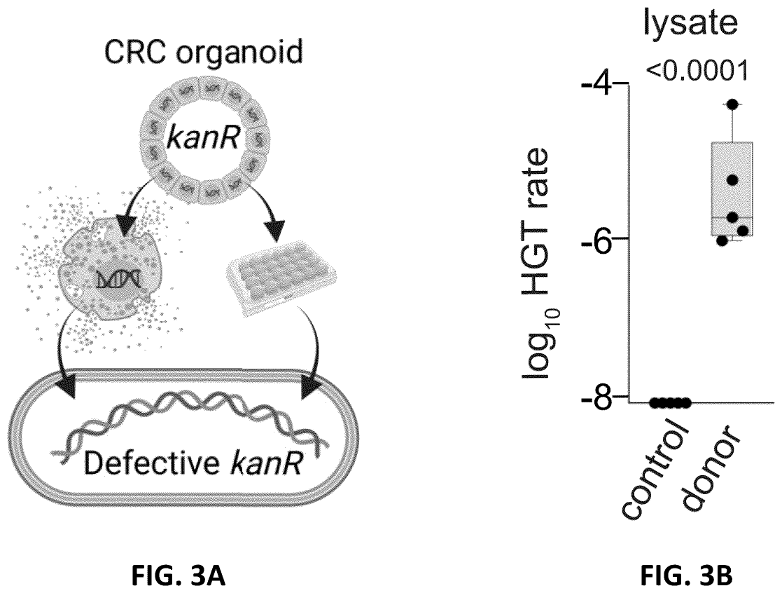

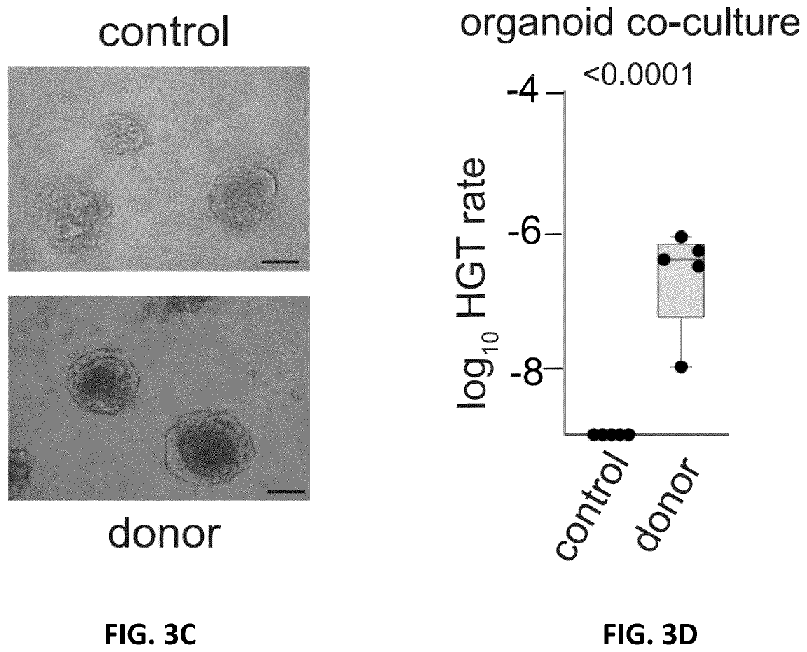

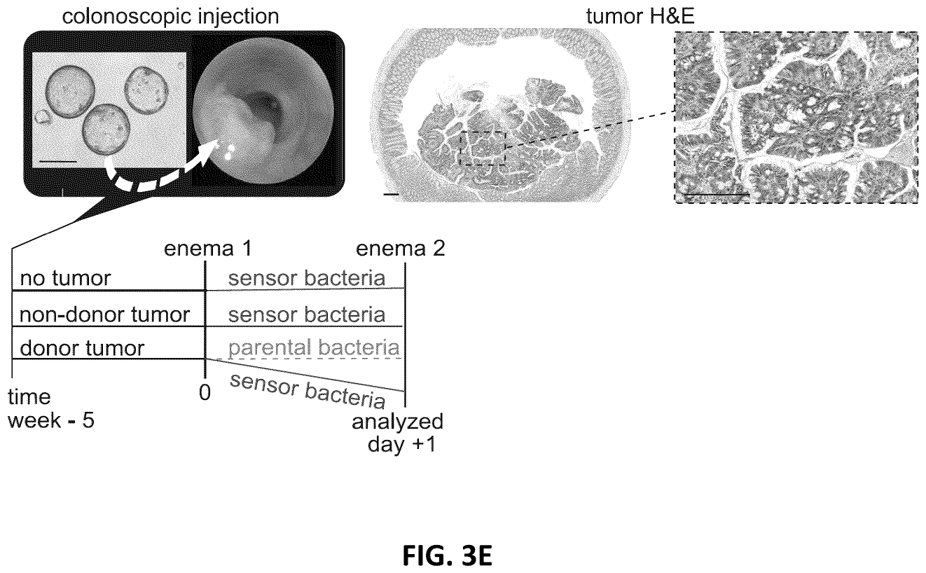

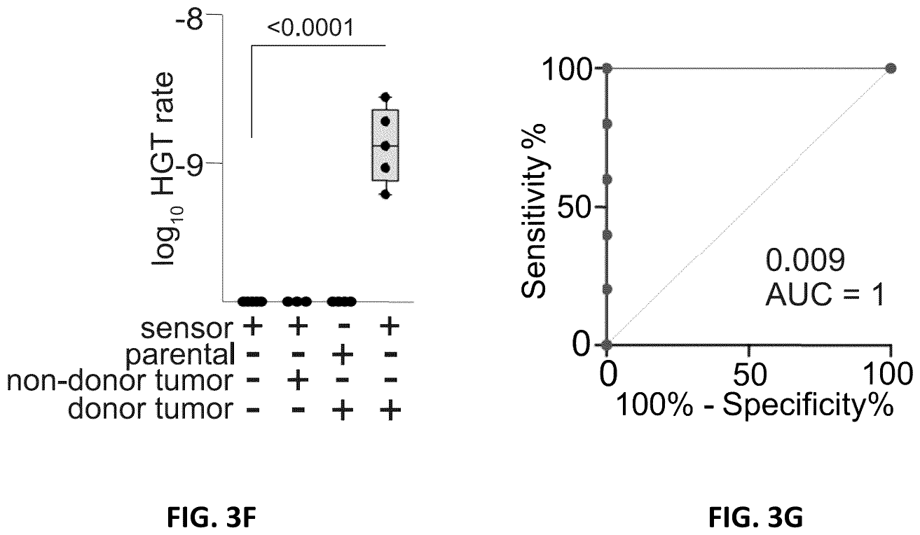

In contrast, living biosensors (e.g., competent sensor bacteria) can uptake target nucleic acid (e.g., DNA or RNA) without preprocessing or purification. Additionally, sensor bacteria can enable DNA detection to work in complex in vivo sites such as gut lumen (FIG. 3E). Living biosensors (e.g., sensor bacteria) can provide built-in signal amplification. Each cell contains multiple membrane receptors for nucleic acid binding and uptake, and a single detection event can activate the entire cell's worth of enzymes. This allows the living biosensors to skip the pre-amplification step that adds time and complexity to many in vitro techniques. Additionally, the living biosensors can enable the entire synthetic biology suite of information processing tools, which opens the door to a far greater range of future analyses and outputs than the in vitro system. While many in vitro biosensors require lab equipment and trained personnel, living biosensors can perform many of these tasks as their own self-sufficient micro-laboratories. As disclosed herein, living biosensors can be used to detect specific target nucleic acids for point-of-care diagnostics.

Provided herein are biosensors (e.g., engineered bacteria) for the detection of target nucleic acids (e.g., DNA or RNA) in a sample. For example, a biosensor can be a composition comprising a bacterium (e.g., A. baylyi), where the bacterium comprises a competence system and an output construct encoding an output signal. In some embodiments, the biosensor is A. baylyi, Bacillus subtilis, Streptococcus pneumoniae, Neisseria gonorrhoeae, Haemophilus influenzae, Vibrio natriegens, Vibrio cholera, or Streptococcus sanguinis. In some cases, S. pneumoniae, N. gonorrhoeae, H. influenzae, or V. cholera are attenuated. In some cases, the biosensor includes a clustered regularly interspaced short palindromic repeat (CRISPR)/Cas system. For example, the CRISPR/Cas system can include an effector nuclease and a spacer directed to a non-targeted nucleic acid (e.g., non-mutated sequence). In some embodiments, the competence system comprises a homologous recombination construct, where a first homology arm is substantially complementary to a sequence upstream of the target nucleic acid and a second homology arm is substantially complementary to a sequence downstream of the target nucleic acid. In some embodiments, the output signal is repressed by a repressor. For example, the output signal can be an antibiotic resistance polypeptide, a fluorescent polypeptide, a bioluminescent polypeptide, a polypeptide that causes a color change, an ultrasound contrast agent polypeptide, a lateral flow assay, or an activated split enzyme.

### Horizontal Gene Transfer (HCT)

Horizontal gene transfer (HGT) is the exchange of genetic material between organisms outside vertical, “parent to offspring” transmission. HGT is common between microbes (12). It may also occur from microbes into animals and plants (13) and, in the opposite direction, from eukaryotes to prokaryotes (14). One example of HGT is natural competence.

Competent bacteria have the ability to alter its genome by integrating extracellular DNA (e.g., a target nucleic acid) from its environment in a process called transformation. Competence can be natural competence, a genetically specified ability of bacteria which occurs under natural conditions, or induced or artificial competence, which arises when cells in laboratory cultures are treated to make them transiently permeable to DNA.

In some embodiments, the bacterium is naturally competent. For example, A. Baylyi is a competent bacteria that can uptake “raw” unpurified DNA as well as biochemically purified DNA. In some bacteria, a competence regulator can be over-expressed (e.g., ComK in B. subtilis, or TfoX and QstR in V. cholera and V. natriegens), as described in Specht D A, et al., Efficient natural plasmid transformation of Vibrio natriegens enables zero-capital molecular biology. PNAS Nexus. 2024 Feb. 13;3 (2): pgad444. doi: 10.1093/pnasnexus/pgad444, which is incorporated by reference in its entirety. In other cases, the bacterium can be artificially induced to become competent. For example, the bacteria can be subjected to techniques known in the art to become permeable (e.g., transiently or permanently) to DNA (e.g., cell-free DNA). In some embodiments, the competence system of the bacteria comprises a type IV pili.

### CRISPR-Cas System

In some embodiments, the engineered living biosensor (e.g., sensor bacteria) comprises endogenous CRISPR/Cas system that includes a spacer directed to a non-target nucleic acid. In some embodiments, the non-targeted nucleic acid can be a non-mutated gene or a wild-type gene. In some embodiments, CRISPR/Cas system can be used to target a specific sequence (e.g., specific mutation). In some cases, CRISPR/Cas system can recognize single-base mutation. For example, the CRISPR/Cas system can enable the biosensor to differentiate KRAS wild-type sequence from KRASG12D sequence in a cancer cell. In some embodiments, upon recognition of the non-target nucleic acid in the sample, the CRISPR/Cas system initiates degradation of the non-target nucleic acid, such that only the target sequence will be detected. For example, the engineered biosensor can have a competence system wherein the competence system includes a complement sequence to a known mutated sequence (e.g., KRASG12D sequence) and a CRISPR/Cas system spacer directed to a wild-type sequence (e.g., KRAS wild-type sequence), wherein CRISPR/Cas system degrades the wild-type sequence and only the mutated sequence (e.g., KRASG12D sequence) is detected.

A CRISPR/Cas system can include a repetitive sequence found within a CRISPR locus (naturally occurring in a bacterial genome or plasmid) that are interspersed with the spacer sequences. A CRISPR repeat sequence can bind to a Cas protein (e.g., any of the Cas proteins known in the art). One skilled in the art would be able to infer the CRISPR repeat sequence of a corresponding Cas protein if the sequence of the associated CRISPR locus is known. A CRISPR repeat sequence can include about 15 to about 36 nucleotides (e.g., about 16, 17, 18, 19, 20, 21, 22, 23, 24, 25, 26, 27, 28, 29, 30, 31, 32, 33, 34, or 35 nucleotides). In some embodiments the CRISPR repeat sequence can include about 20 to about 36 nucleotides, about 25 to about 36 nucleotides, about 30 to about 36 nucleotides, about 15 to about 25 nucleotides, or about 20 to about 25 nucleotides. A CRISPR repeat sequence can be a CRISPR repeat sequence for a Cas protein that is capable of processing its own pre-crRNA in to mature crRNA (e.g., processing natural arrays without tracrRNA), for example Cas 12a (Cpf1) or Cas13a (C2c2). For example, the repeat sequence can be for FnCpf1, AsCpf1, or LbCpf1. In some embodiments, the CRISPR system can comprise (a) a guide RNA comprising CRISPR RNA (crRNA) and tracr RNA that binds to a target sequence; and (b) a Cas polypeptide comprising a crRNA processing domain, a target coordination domain, and one or more enzymatic cleavage domain. In some cases, the CRISPR/Cas system includes I-A, I-B, I-C, I-D, I-E, I-F1, I-F2, I-F3, I-G, II-A, II-B, II-C1, II-C2, III-A, III-B-, III-C, III-D, III-E, III-F, IV-A, IV-B, IV-C, V-A, V-B1, V-B2, V-C, V-D, V-E, V-F1, V-F2, V-F3, V-G, V-U1, V-U2, V-U4, V-U5, VI-A, VI-B1, VI-B2, VI-C, or a VI-D CRISPR-Cas system. Non-limiting examples of Cas nucleases include, without limitation, Cas1, Cas2, Cas3, Cas9, Cas10, Cas12, Cas13, Cas14, or Cpf1.

### Repressors/Output

In some embodiments of the methods described herein, the engineered biosensor (e.g., sensor bacteria) comprises a repressor gene (e.g., tetracycline repressor gene) that can repress the expression of an output gene construct (e.g., antibiotic resistance gene). In some cases, the repressor gene can be inserted between the homology arms in the biosensor. In some embodiments, the repressor is a polypeptide repressor, an RNA-based repressor, or a CRISPR/Cas system-based repressor. For example, the polypeptide repressor can be a tetracycline repressor (tetR), a lac repressor (lacI), a CI repressor, a PhIF repressor, a IcaRA repressor, an AmtR repressor, a BetI repressor, a SrpR repressor, an Orf2 repressor, a BM3R1 repressor, a ButR repressor, a PhIF repressor, an AmeR repressor, a QacR repressor, a LmrA repressor, a PsrA repressor, a HlyIIR repressor, a McbR repressor, a ScbR repressor, a TarA repressor, a LitR repressor, a HapR repressor, or a SmcR repressor. An RNA-based repressor can be a small RNA-based repressor, a toehold repressor, a 3-way junction repressor, or a pT181dual transcription/translational repressor.

In some embodiments of the methods described herein, the engineered biosensor (e.g., sensor bacteria) comprises an output gene that is repressed (e.g., constitutively or conditionally) by a repressor gene. For example, a repressor gene (e.g., tetR) located between the homology arms on a biosensor (e.g., A. baylyi) genome can repress the expression of an output signal (e.g., kanamycin resistance). In some embodiments, homologous recombination of the target nucleic acid sequence of a sample with the homology arms in the biosensor excises and eliminates the repressor gene. The elimination of the repressor gene can drive the expression of the output gene. For example, the recombination with the KRASG12D target nucleic acid can excise the tetR gene from the biosensor gene, thus, relieving the repression of kanamycin resistance gene in a biosensor.

In some cases, the biosensor comprises a repressor gene that is operably linked to an output gene construct. An output gene construct can encode an output signal. For example, an output construct (e.g., output gene) can be operably linked to a P_LtetO-1 promoter to express the repressor gene tetR. In some embodiments, the output signal is repressed by DNA looping with a repressor binding site located outside a CRISPR-targeted transposon (CAST). In some cases, the CAST recognizes the target nucleic acid. In some embodiments, upon recombination, the transposon relocates itself to the target site and relieves the repression by moving the output gene construct away from the repressor binding site to allow expression of the output gene.

In some embodiments, an output signal can be an antibiotic resistance, a fluorescent polypeptide, a polypeptide which reacts with a substrate to produce luminescence, a polypeptide that causes a color change, an ultrasound contrast agent, a lateral flow assay, or an activated split enzyme. For example, the antibiotic resistance output signal can be resistance to kanamycin, ampicillin, chloramphenicol, spectinomycin, puromycin, gentamicin, hygromycin, or bleomycin. Non-limiting examples of fluorescent polypeptide include green fluorescent protein (GFP), red fluorescent protein (RFP), or yellow fluorescent protein (YFP). Non-limiting examples of bioluminescent polypeptide includes firefly luciferase, renilla luciferase, acquorin, or gaussian luciferase.

In embodiments, the output signal can be an activated split enzyme. For example, the split enzyme can be a split intein, for example as described in Yao Z, et al. Split Intein-Mediated Protein Ligation for detecting protein-protein interactions and their inhibition. Nat Commun. 2020 May 15;11 (1): 2440. doi: 10.1038/s41467-020-16299-1, which is incorporated by reference in its entirety. In such a case, the target DNA would reconstitute the split intein, which then splices together the desired output protein. In some cases, each half of a split enzyme is fused to nuclease-inactivated CRISPR/Cas effectors. The CRISPR/Cas effectors direct the split enzyme to adjacent regions on a target nucleic acid sequence causing homologous recombination. In some embodiments, bringing the split haves of the enzyme in close proximity restores its activity, leading to signal output.

In some embodiments, the output of the methods disclosed herein can be detected by lateral flow assay. In some embodiments, a lateral flow assay uses two or more antibodies to detect the output signal in a biosensor (e.g., sensor bacteria). In some embodiments, signaling molecule secreted by the engineered biosensor can be engineered to express specific distinguishable tags (e.g., His tags and FLAG tags) to enable specific detection by capture and detection antibodies on a lateral flow assay test strip. For example, the lateral flow assay test strip can be immobilized with streptavidin at the test line that can bind free biotinylated anti-6×His. The anti-6×His on the test line can then bind the 6×His epitopes on the Hcp-6×His-FLAG signaling molecule. In some embodiments, the antibody is attached to one or more gold nanoparticles. In some embodiments, the size of the gold nanoparticle is from about 40 nm to about 500 nm (e.g., from about 40 nm to about 450 nm, from about 50 nm to about 400 nm, from about 100 nm to about 350 nm, from about 150 nm to about 300 nm, from about 200 nm to about 250 nm, about 40 nm, about 100 nm, about 150 nm, about 200 nm, about 250 nm, about 300 nm, about 350 nm, about 400 nm, about 450 nm, or about 500 nm). In some embodiments, the 6×His and the FLAG tags are at the C-terminus on the Hep molecule and are separated by a flexible linker (e.g., an 8×Gly flexible linker). In some embodiments, additional FLAG tags can be added at the N-terminus and/or internal to the Hop molecule.

### Methods of Use

Provided herein are methods of detecting a target nucleic acid in a sample, where the method includes a) administering to the sample an effective amount of a composition comprising a bacterium (e.g., biosensor bacteria), and b) detecting an output signal. In some embodiments, the bacterium (e.g., A. baylyi) is a living biosensor. In some embodiments, the bacterium comprises a competence system and a CRISPR/Cas system. In some cases, the competence system comprises a first homology arm that is substantially complementary to a sequence upstream of the target nucleic acid and a second homology arm that is substantially complementary to a sequence downstream of the target nucleic acid. In some embodiments, the biosensor can comprise a CRISPR/Cas system including an effector nuclease and a spacer directed to a non-target nucleic acid (e.g., non-mutated gene or wild-type gene). In some embodiments, the detection of the target nucleic acid (e.g., KRASG12D mutation) in the sample can relieve the repression of the output gene, thus turning on the output signal (e.g., antibiotic resistance expression, a fluorescent polypeptide, a signal detectable by a lateral flow assay). In some embodiments, the detection of a non-target nucleic acid (e.g., KRAS WT) initiates degradation of the non-target nucleic acid and maintenance of the repression of the output gene construct. In some embodiments, the output signal is encoded by an output gene construct. The methods described herein can be used to detect nucleic acid (e.g., DNA or RNA) in vivo as well as in vitro.

The engineered biosensor circuit is modular, and thus can be adapted to environmental samples. Samples such as air sample, water sample (e.g., waste-water sample, surface water sample), soil samples (e.g., crop soil sample), a dust sample, or an agricultural product sample. can be analyzed using the methods described herein to monitor public health, pollution levels, infection, microbial quality, sample quality, epidemiological surveillance, or compliance monitoring. The affordability and the case of use of biosensor such as engineered A. baylyi provides a tool to assess the environmental health and identify potential risks to public health.

Further, the engineered biosensors of the disclosure can be adapted and used to detect a condition in a mammal (e.g., a human). In some embodiments, a biological sample is collected from a mammal (e.g., human) and the methods of the disclosure are performed on the biological sample. For example, a biological sample can be a cancer cell, a tissue sample, a body fluid (e.g., blood or plasma), a fecal sample, a body secretion, a urine sample, a sputum, or a hair sample.

In some embodiments, the methods described herein can be used to detect a target nucleic acid (e.g., oncogene, a viral gene, a bacterial gene) in a mammal that has or is suspected of having a cancer or an infectious disease, or to determine whether the infectious disease is an antimicrobial resistant infection. In some embodiments, the target nucleic acid (e.g., DNA or RNA) is in a sample. For example, the target nucleic acid can be an oncogene, a bacterial gene, or a viral gene. In some cases, the sample can be a biological sample or an environmental sample. For example, the biological sample can be a sample from a mammal (e.g., a human). The biological sample can be a cancer cell, a tissue sample, a body fluid (e.g., blood or plasma), a fecal sample, a body secretion, a urine sample, a sputum, or a hair sample. The environmental sample can include an air sample, a water sample, a dust sample, an agricultural product sample, a sewage sample, a plant sample, a sewage sample, or a soil sample.

In some embodiments of the methods described herein, the mammal has or is suspected of having a cancer. For example, the cancer can be a colorectal cancer (CRC), a pancreatic cancer, head and neck squamous cell carcinoma, a renal carcinoma, a breast cancer, a lung cancer, a prostate cancer, a glioma, a melanoma, a keratinocyte cancer, an ovarian cancer, a liver cancer, a kidney cancer, a bladder cancer, a thyroid cancer, a sarcoma, a stomach cancer, a cervical cancer, an endometrial cancer, an esophageal cancer, a thymoma, a soft tissue sarcoma, a bone cancer, a testicular cancer, a penile cancer, a gallbladder cancer, a uterine sarcoma, an adrenal gland cancer, an ampullary cancer, a hepatic angiosarcoma, a nasal, a paranasal sinus cancer, leukemia, a lymphoma, a myeloma, a myelodysplastic syndrome, or a myeloproliferative neoplasm.

In some embodiments of the methods described herein, the engineered biosensors of the disclosure can detect oncogenes such as adenomatous polyposis coli (APC), mutL homolog 1 (MLH1), mutS homolog 2 (MSH2), mutS homolog 6 (MSH6), postmeiotic segregation increased 2 (PMS2), transforming growth factor-beta receptor 2 (TGFBR2), phosphatase and tensin homolog (PTEN), Kirsten rat sarcoma virus (KRAS), Tumor protein p53 (TP53), Mothers against decapentaplegic homolog 4 (SMAD4), or V-Raf murine sarcoma viral oncogene homolog B1 (BRAF).

In some embodiments, the detection methods described herein can be used on a biological sample from a mammal (e.g., human) who has an increased risk of developing cancer (e.g., breast cancer, lung cancer, colon cancer, prostate cancer). For example, a human may have a family history and/or personal history of cancer. In some cases, the cancer is characterized by a mutated KRAS gene, a mutated TP53, a mutated SMAD4, or a mutated BRAF gene. For example, the mutated KRAS gene can comprise a G12D, a G13, or a Q61 mutation; the mutated TP53 gene can comprise a R175 or a R248 mutation; the mutated SMAD4 gene can comprise a R361 or a R265 mutation; the mutated BRAF gene can comprise a BRAFV600E mutation.

In some cases, the detection methods described herein can be used on a biological sample from a mammal (e.g., a human) who has undergone previous treatments (e.g., cancer treatments, infectious disease treatments). For example, cancer treatment can include chemotherapy alkylating agents (e.g., nitrogen mustards such as mechlorethamine, cyclophosphamide, melphalan, chlorambucil, ifosfamide and busulfan; nitrosoureas such as N-Nitroso-N-methylurea (MNU), carmustine (BCNU), lomustine (CCNU) and semustine (MeCCNU), fotemustine and streptozotocin; tetrazines such as dacarbazine, mitozolomide and temozolomide; aziridines such as thiotepa, mytomycin and diaziquone (AZQ); cisplatins and derivatives such as cisplatin, carboplatin and oxaliplatin; and non-classical alkylating agents such as procarbazine and hexamethylmelamine), anti-metabolites (e.g., anti-folates such as methotrexate and pemetrexed; fluoropyrimidines such as fluorouracil and capecitabine; deoxynucleoside analogues such as cytarabine, gemcitabine, decitabine, Vidaza, fludarabine, nelarabine, cladribine, clofarabine and pentostatin; and thiopurines such as thioguanine and mercaptopurine), anti-microtubule agents (e.g., vinca alkaloids such as vincristine, vinblastine, vinorelbine, vindesine, and vinflunine; and taxanes such as paclitaxel, docetaxel, and cabazitaxel; podophyllotoxin; etoposide; and teniposide), topoisomerase inhibitors (e.g., topoisomerase I inhibitors such as irinotecan and topotecan; and topoisomerase II inhibitors such as etoposide, doxorubicin, mitoxantrone, teniposide, novobiocin, merbarone, and aclarubicin), and cytotoxic antibiotics (e.g., anthracyclines such as doxorubicin, daunorubicin, epirubicin, idarubicin, pirarubicin, aclarubicin, and mitoxantrone; bleomycins; mitomycin C; mitoxantrone; and actinomycin). In some embodiments, additional cancer therapy includes immune checkpoint blockade therapy. For example, the immune checkpoint blockade therapy can include PD-1 inhibitors, PD-L1 inhibitors, CTLA-4 inhibitors, and/or LAG-3 inhibitors. The PD-1 and PD-L1 inhibitors can include nivolumab, pembrolizumab, atezolizumab, avelumab, durvalumab, cemiplimab, dostarlimab, retifanlimab, toripalimab, vopratelimab, spartalizumab, camrelizumab, sintilimab, tislelizumab, INCMGA00012, AMP-224, AMP-514, acrixolimab, KN035, cosibelimab, AUNP12, CA-170, and/or BMS-986189. The CTLA-4 inhibitors can include ipilimumab and/or tremelimumab. The LAG-3 inhibitors can include relatlimab.

In some embodiments, the engineered biosensors can detect infectious disease such as a microbial infection. In some embodiments, the microbial infection is caused by a bacterium, a virus, a fungus, a parasite, or protozoa. For example, a bacterial infection can include strep throat, tuberculosis, urinary tract infections (UTI), pneumonia, gonorrhea, meningitis, salmonella infection, Lyme disease, or cholera. A viral infection can include influenza, common cold, COVID-19, HIV/AIDS, hepatitis (A, B, or C), chickenpox, measles, herpes, or Ebola. A fungal infection can include athlete's foot, ringworm, yeast infection, pneumocystis pneumonia, aspergillosis, candidemia, cryptococcosis, histoplasmosis, or coccidioidomycosis. Non-limiting examples of parasitic infections include malaria, giardiasis, toxoplasmosis, trichomoniasis, cryptosporidiosis, amoebiasis, chagas disease, hookworm infection, or filariasis.

In some embodiments, the engineered biosensors can detect an antimicrobial resistant infection. Non-limiting examples of antimicrobial resistant infection includes methicillin-resistant Staphylococcus aureus (MRSA) infection, multi-drug resistant tuberculosis (MDR-TB), carbapenem-resistant enterobacteriaceae (CRE) infection, fluoroquinolone-resistant campylobacter infection, vancomycin-resistant enterococcus infection, or extended-spectrum beta-lactamase-producing enterobacteriaceae infection.

A “effective amount” is an amount sufficient to effect beneficial or desired results. For example, an effective amount is one that achieves a desired effect, e.g., an amount necessary to detect a target nucleic acid in a sample (e.g., biological sample or environmental sample). An effective amount can be administered in one or more administrations, applications, or dosages. An effective amount (i.e., an effective dosage) of the biosensor (e.g., sensor bacteria) depends on the type of the sample and the biosensor. The skilled artisan will appreciate that certain factors may influence the dosage and timing required to effectively detect a target nucleic acid in a sample, including, but not limited to, the type of the sample, severity of the disease or disorder, previous treatments, the general health and/or age of the subject, and other diseases present. Various factors can influence the actual amount used for a particular application.

### EXAMPLES

The invention is further described in the following examples, which do not limit the scope of the invention described in the claims.

### Example 1: Engineered Bacteria Detect Tumor DNA

### Results

### Engineering Cancer Cell Lines, Organoids, and Sensor Bacteria

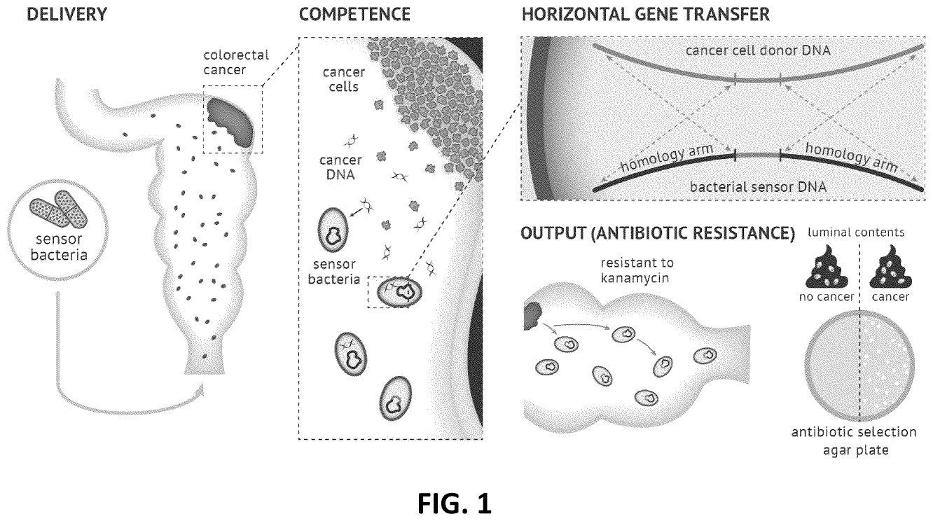

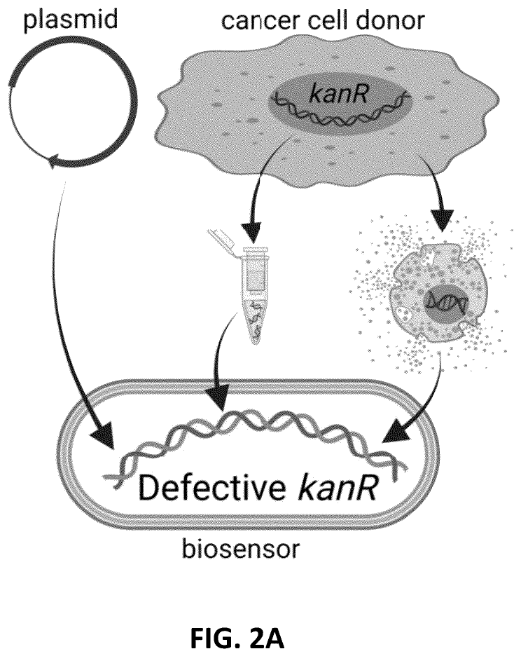

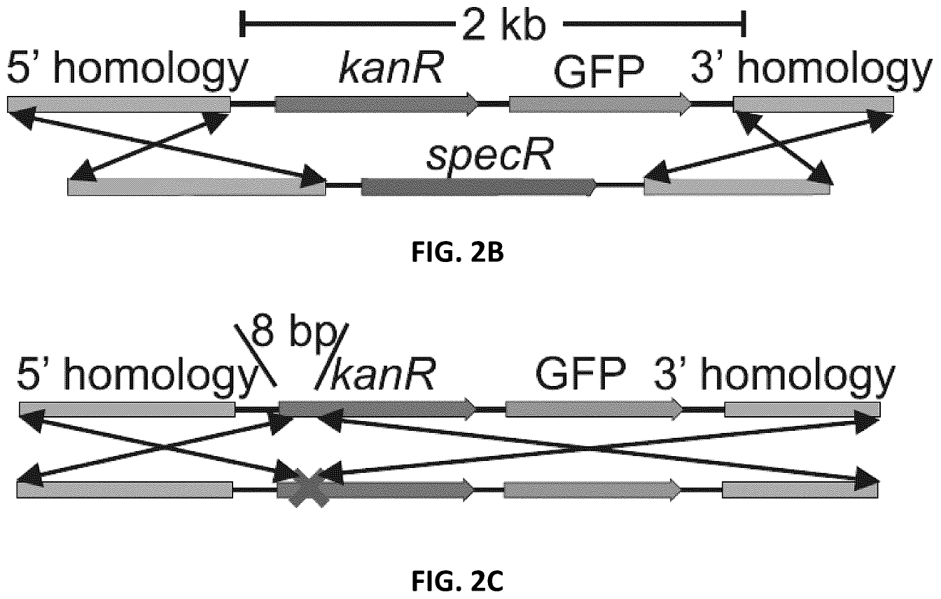

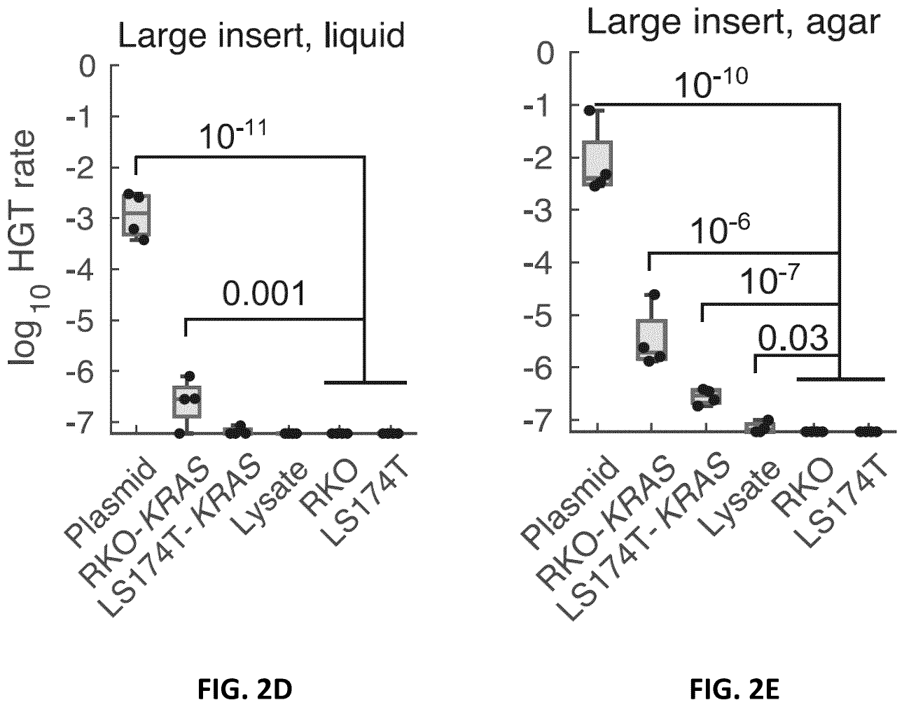

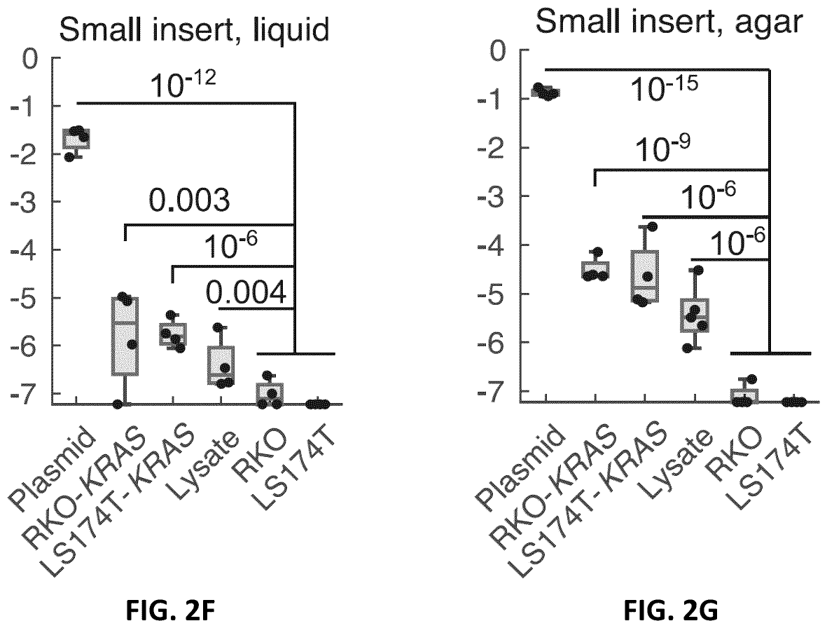

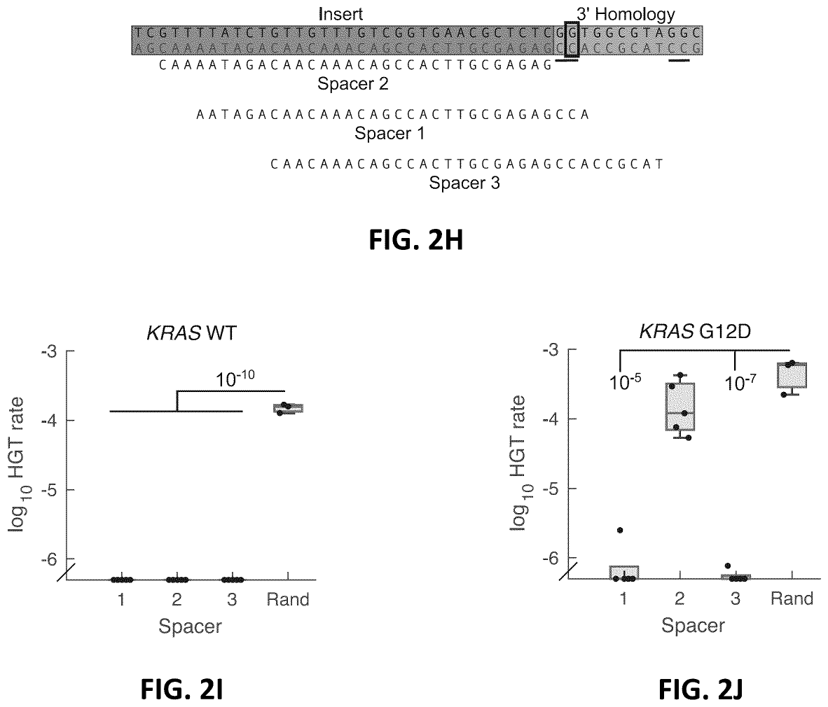

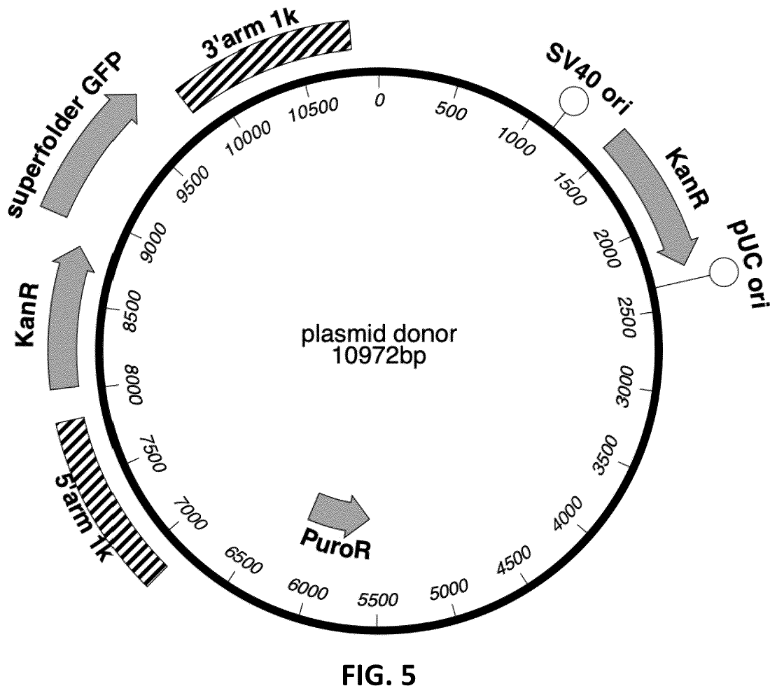

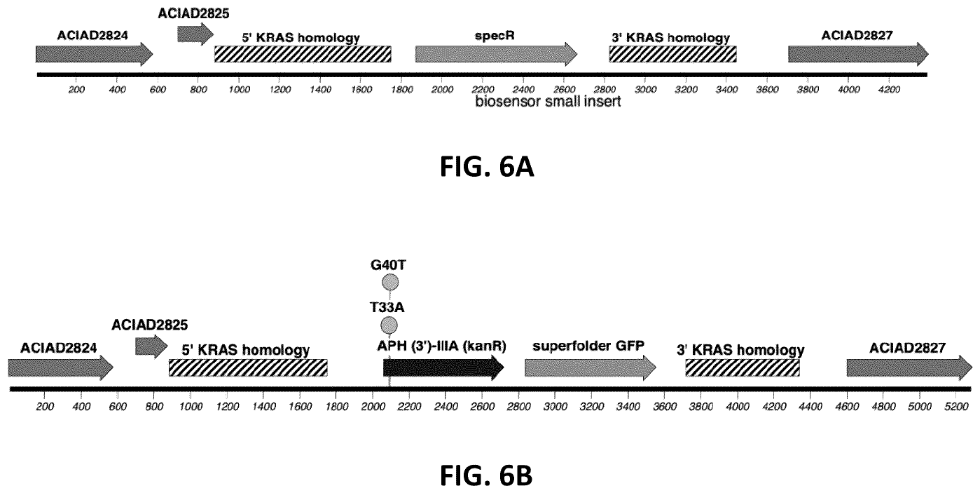

To test if bacteria could detect specific mammalian DNA, transgenic donor human cancer cells lines were generated with a kanamycin resistance gene (kanR) inside KRAS homology arms (FIGS. 1A-1C, FIGS. 2A-2C, FIG. 5, and FIG. 6). The kanR donor cassette was transduced into three conventional human colorectal cancer (CRC) cell lines with differing background genetic alterations: i) RKO is microsatellite instability high (MSI-H), ii) BRAFV600E, iii) LS174T is MSI-H, KRASG12D, and iv) SW620 is microsatellite stable (MSS), KRASG12V, and two human CRC organoid lines: i) RAH057T is MSS, KRASG12D and RAH038T is MSI-H, BRAFV600E using a lentiviral vector. To construct the sensor bacteria, a complementary landing pad was inserted with KRAS homology arms into Acinetobacter baylyi. A “large insert” design, where 2 kb of donor cassette must transfer (FIGS. 2A-2B; FIG. 6A), and a “small insert” design, where only 8 base pairs (bp) must transfer to repair two stop codons (FIG. 2C, FIG. 6B) were tested. The initial biosensor output was measured as growth on kanamycin plates (FIG. 2 and FIG. 6).

Detection of Cell-Free DNA from Cancer Cell Lines

The “large insert” and the “small insert” designs were tested using various donor DNA sources, both in liquid culture and on solid agar (FIG. 2A). The “large insert” biosensors detected donor DNA from purified plasmids and genomic DNA both in liquid (FIG. 2D) and on agar (FIG. 2E). On agar, they also detected raw, unpurified lysate, albeit at just above the limit of detection (FIG. 2E). The “small insert” design improved detection efficiency by ˜10-fold, reliably detecting even raw lysate (FIGS. 2F-2G). Across conditions, detection on solid agar was more efficient than in the liquid culture. These experiments confirmed that the biosensors did not require DNA purification.

Mutations in codon 12 of KRAS occur in 27% of CRC tumors (F. André et al., Cancer Discov. 7:818-831 (2017)), accounting for 72% of all CRC KRAS mutations (W. Li et al., Int. J. Clin. Exp. Pathol. 12:957-967 (2019)), and are common in solid tumors generally (P. Priestley et al., Nature. 575:210-216 (2019)). To test whether sensor bacteria could discriminate between wild-type and mutant KRAS (KRASG12D), A. baylyi's endogenous type I-F CRISPR-Cas system was used. A donor RKO cell line was transduced with kanR-GFP (green fluorescent protein) donor cassette flanked by wild-type KRAS or KRASG12D flanking sequences. Next, three CRISPR spacers targeting the wild-type KRAS sequence at the location of the KRASG12D mutation were designed using the A. baylyi protospacer-adjacent motif (PAM) (FIG. 2H). These spacers were inserted as single-spacer arrays into a neutral locus in the “large insert” A. baylyi sensor genome.

The sensor bacteria should reject the wild-type KRAS through CRISPR-mediated DNA degradation but allow integration of the KRASG12D sequence. Two of the three spacers blocked transformation by both wild-type and mutant DNA (FIGS. 2I and 2J). However, spacer 2, for which the KRASG12D mutation eliminated the PAM site, selectively permitted only KRASG12D donor DNA (FIGS. 2I and 2J). The other common mutations in codon 12 of KRAS all eliminated this PAM as well. Thus, sensor A. baylyi can be engineered to detect a mutational hotspot in the KRAS gene with single-base specificity.

Detection of Cell-Free DNA from Tumorigenic Organoid Lines

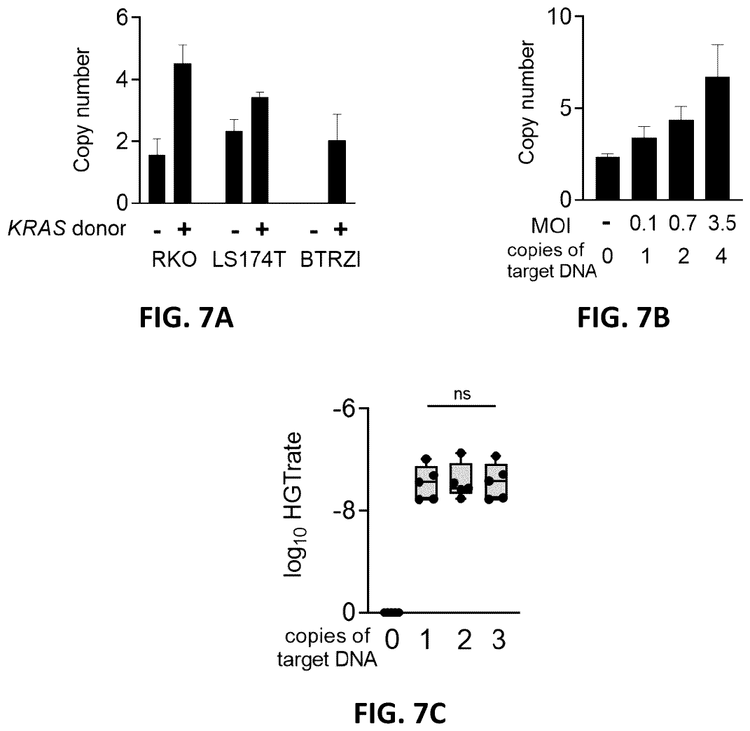

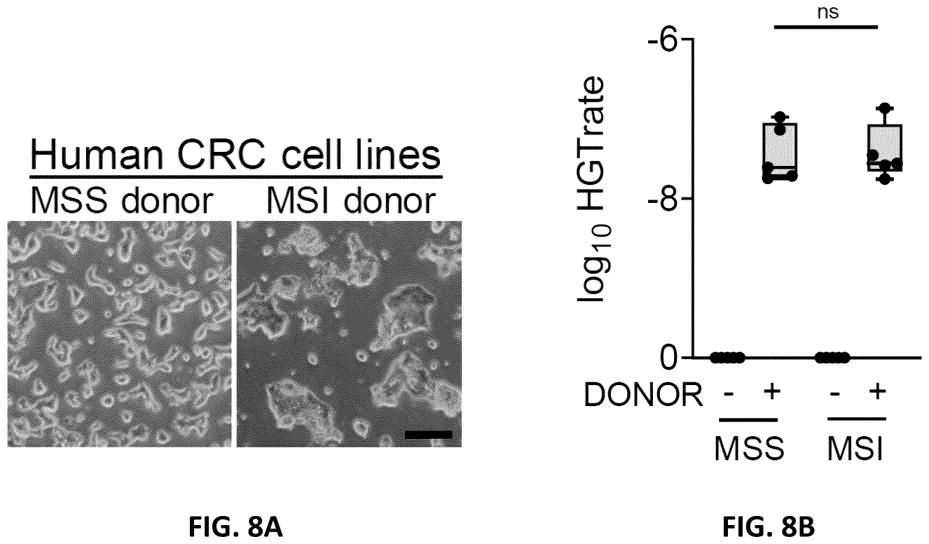

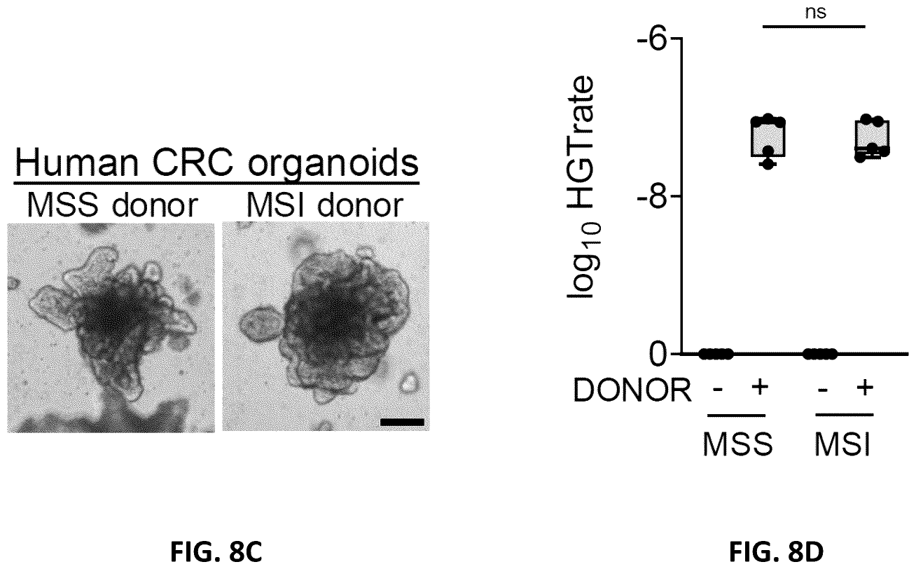

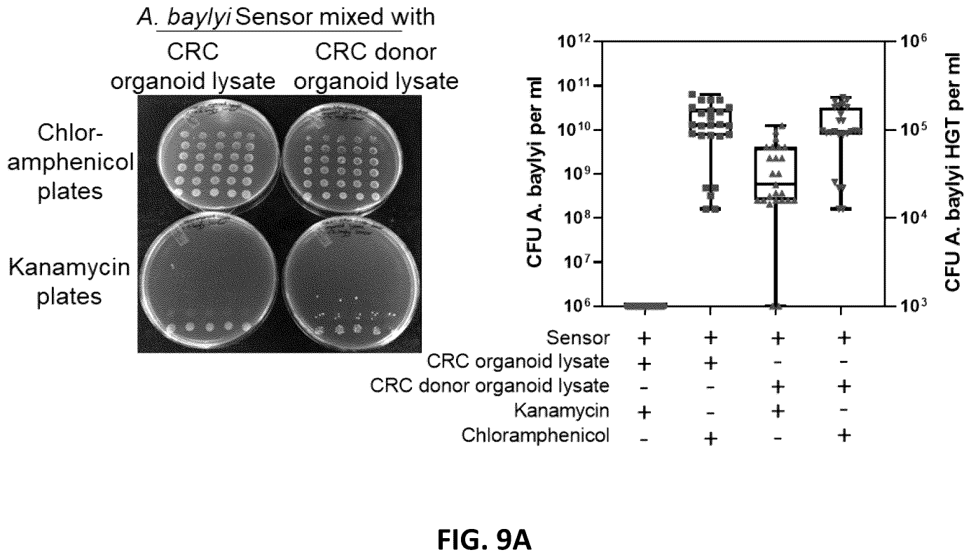

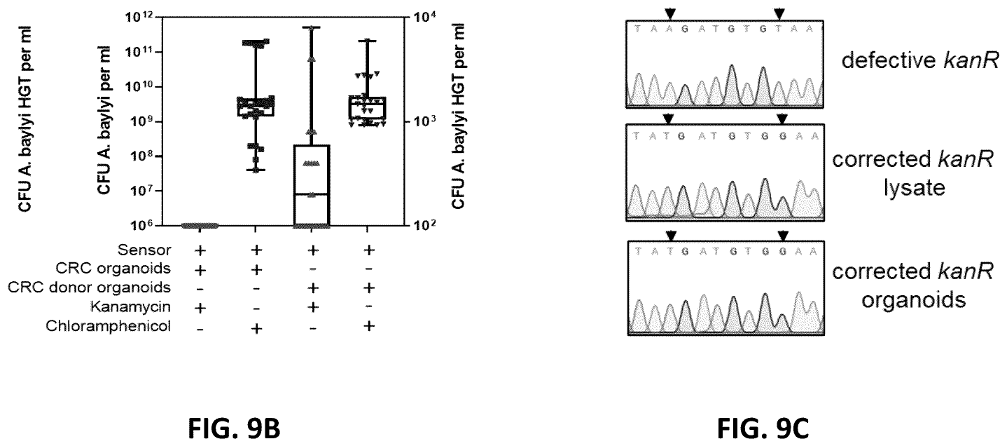

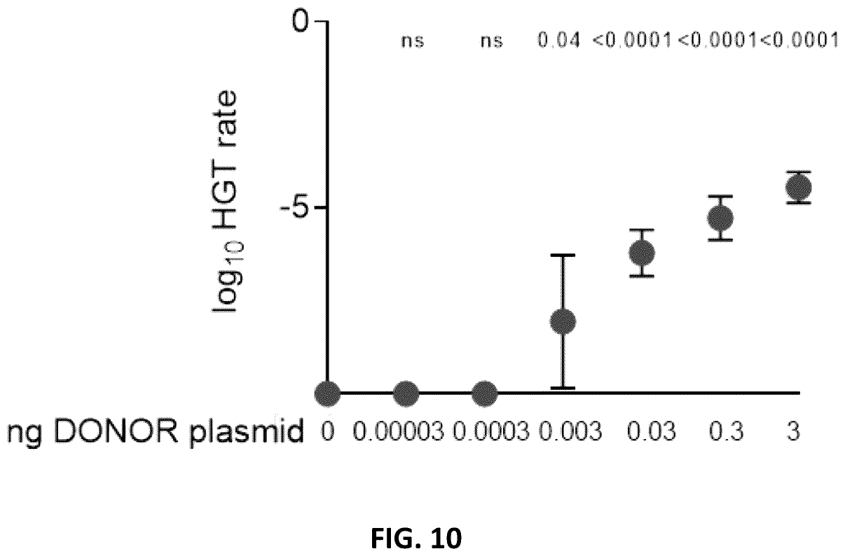

Next, the sensor and the donor constructs were evaluated in organoid culture (FIG. 3A). Mouse colorectal cancer (CRC) organoids with Braf^{V600E}, Tgfbr2^{Δ/Δ}, Rnf43^{Δ/Δ}, Znrf3^{Δ/Δ}, and p16Ink4a^{Δ/Δ} mutations (BTRZI) were transduced with the donor DNA construct to generate donor CRC organoids. The organoid lysate was incubated with the “small insert” A. baylyi biosensors. Using quantitative polymerase chain reaction (qPCR), it was confirmed that the BTRZI organoids contained only two copies of the target donor DNA (FIG. 7). As with the CRC cell lines, the sensor A. baylyi incorporated DNA from donor organoid lysate, but not from control lysates from the parental organoids (FIG. 3, FIG. 8, and FIG. 9A). Next, GFP-expressing sensor A. baylyi were co-cultured with parental or donor organoids for 24 hours on Matrigel. The GFP-expressing sensor bacteria enveloped the organoids (FIG. 3C). After coculture with the donor organoids, the A. baylyi sensor bacteria acquired donor DNA through Horizontal Gene Transfer (HGT) (FIG. 3D and FIGS. 9B and 9C). To estimate the detection limit of the biosensor for target DNA in stool, increasing amounts of donor plasmid were added to a defined mixture of biosensor and stool (5×10⁷ biosensor mixed with 0.017 g per 100 mL of stool slurry). The detection limit was 3 pg of plasmid or 2.7×10⁵ copies of target DNA, for a given incubation volume and time (FIG. 10).

### Detection of Cell-Free Tumor DNA in an Orthotopic Mouse Model of Colorectal Cancer

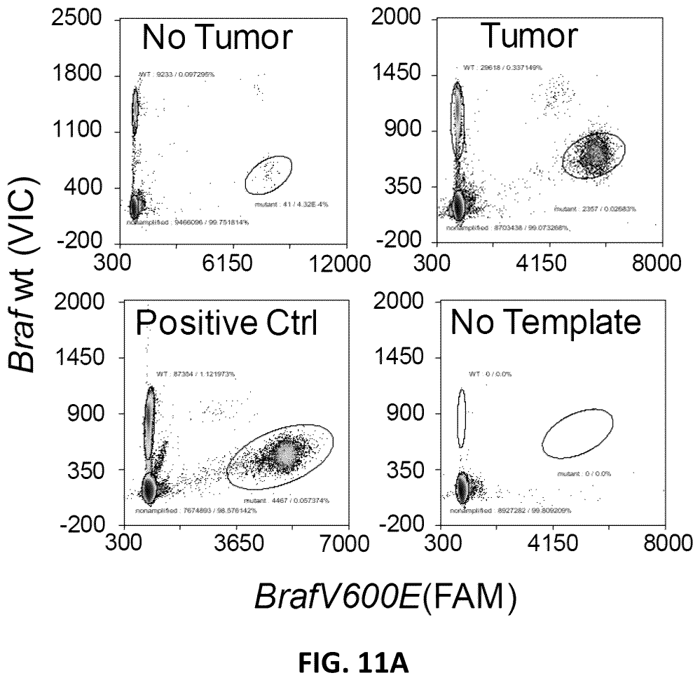

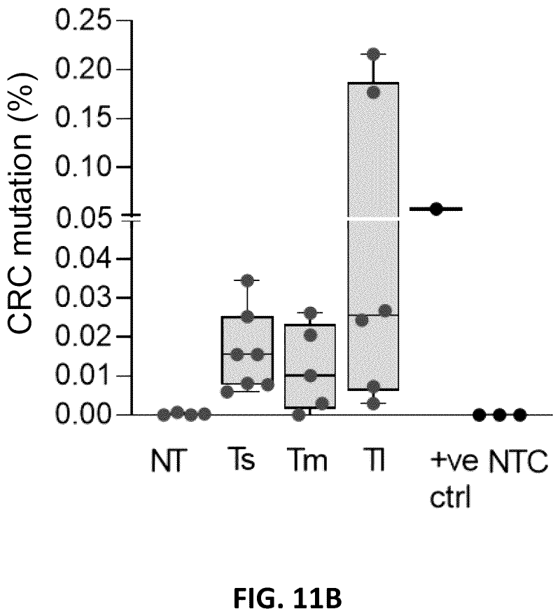

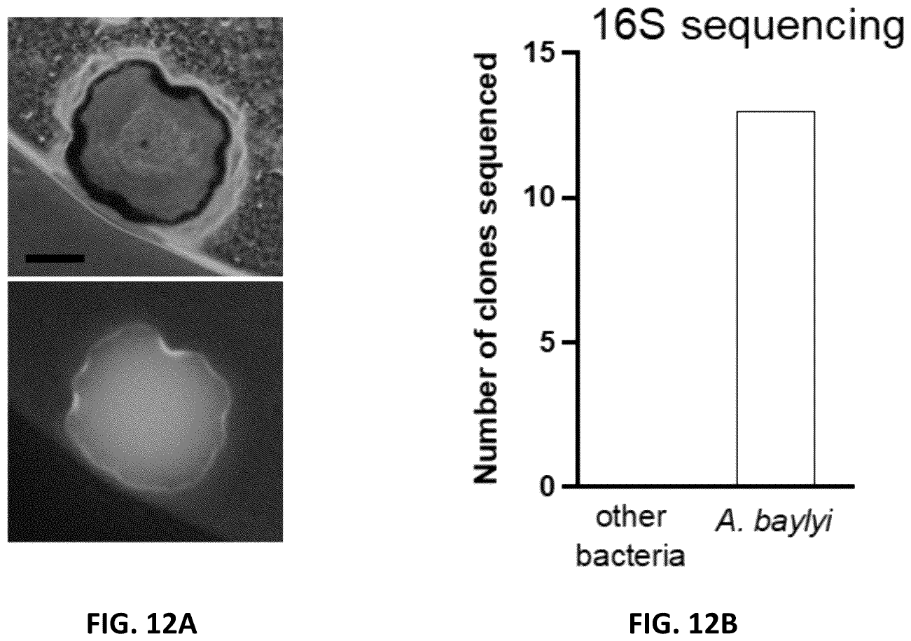

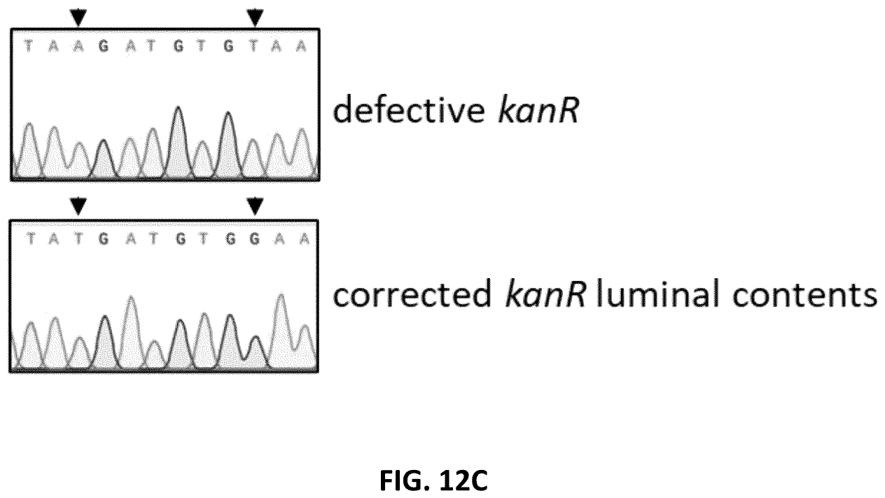

Given that cancer-to-bacterial HGT occurred in vitro and in the presence of stool, the cellular assay for target, CRISPR-discriminated horizontal gene transfer (CATCH) system was tested in vivo. It was first confirmed that the BTRZI orthotopic CRC model released tumoral DNA into the colorectal lumen. Engineered CRC organoids were injected orthotopically, by mouse colonoscopy, into the mouse colon to form colonic tumors. Using digital droplet PCR, Braf mutant tumor DNA level was measured in stools collected from tumor-bearing mice and control mice. The BTRZI model reliably released tumor DNA into the colorectal lumen (FIG. 11).

Next, an orthotopic CRC experiment was conducted (FIG. 3E). Immunodeficient NSG mice were injected with donor or nondonor organoids, or neither. At week 5, once the tumors had grown into the lumen, sensor (or parental) A. baylyi bacteria were delivered twice through rectal enema. The mice were subsequently euthanized and the colorectum was harvested with the luminal effluent plated for analysis. Serial dilutions were then plated on agar with different antibiotic combinations (FIG. 3F).

HGT from the tumors to the biosensors was only detected in donor tumor-bearing mice that were administered sensor bacteria. There was no HGT detected in any control group (FIG. 3F). The resistant colonies were confirmed to be the engineered biosensor strain by antibiotic resistance, green fluorescence, 16S sequencing, and HGT-mediated kanR repair of the individual colonies (FIG. S8). Thus, CATCH successfully discriminated mice with and without CRC in this experimental model (FIG. 3G).

### Detection of Nonengineered DNA

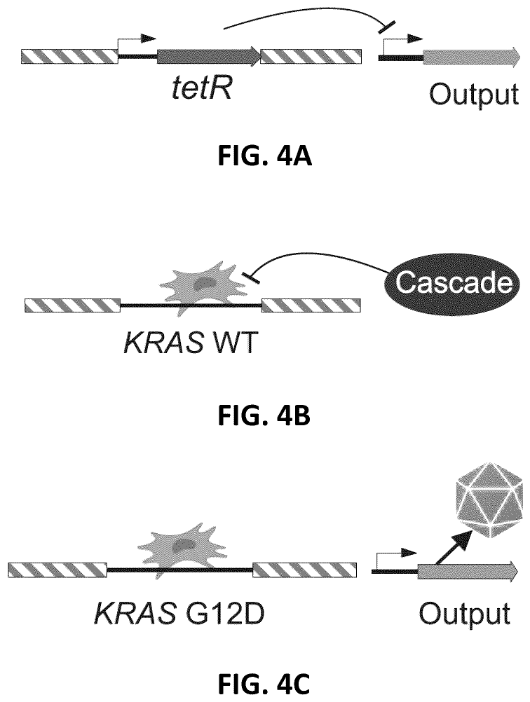

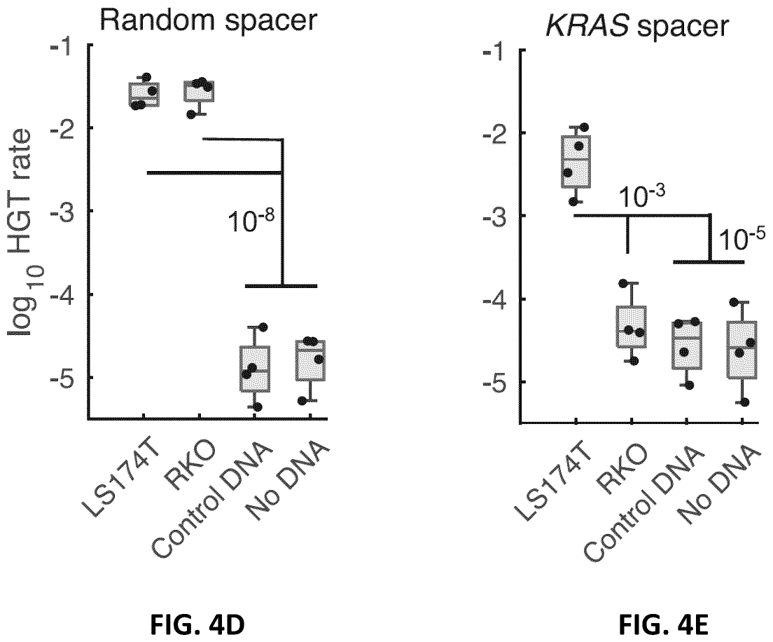

To detect and analyze nonengineered cancer DNA, living biosensors were designed. The tetR repressor gene was inserted between the KRAS homology arms in the biosensor, and in a second locus, an output gene was placed under the control of the P_LtetO-1 promoter (FIG. 4A). Here, the output gene was kanamycin resistance for ease of measurement, but the output gene is arbitrary and exchangeable.

In this design, expression of the output gene was constitutively repressed (FIG. 4A). Upon recombination with the KRAS target DNA, the repressor tetR was deleted from the genome. If the incoming KRAS sequence was wild type at the G12 locus, Cascade, the type I-F CRISPR-Cas effector complex, detected and degraded it (FIG. 4B). However, if the G12 locus was mutated, the PAM site and therefore the CRISPR-Cas targeting were eliminated, and the expression from the output gene was turned on (FIG. 4C).

This natural DNA sensor design was tested in vitro using PCR products from LS174T and RKO genomes as donor DNA. Natural DNA biosensors with a random CRISPR spacer detected DNA sequences from both cell lines (FIG. 4D), and biosensors with the KRAS spacer accurately detected only DNA sequence from LS174T cells, which contain the KRASG12D mutation (FIG. 4E), demonstrating the biosensor detection and discrimination of the natural target DNA.

### Methods

### Bacterial Cell Culture and Cloning to Generate Biosensors

Acinetobacter baylyi ADP1 was obtained from the American Type Culture Collection (ATCC #33305) and propagated in standard LB medium at 30° C. or 37° C. KRAS homology arms were inserted into a neutral genetic locus denoted Ntrl1, replacing the gene remnant ACIAD2826. For the “large insert” design, a spectinomycin resistance gene was placed between the KRAS homology arms. For the “small insert” design, two stop codons were placed near the beginning of the kanR gene of the donor cassette, and the broken cassette was inserted into A. baylyi. CRISPR arrays were inserted into a neutral locus used by replacing ACIAD2186, 2187, and part of 2185. Ectopic CRISPR arrays were driven by a promoter region that included 684 bp from upstream of the first repeat of the endogenous, 90-spacer array.

For natural DNA biosensors, a temperature-sensitive tetR repressor was placed between the KRAS homology arms. An output gene, either kanR or GFP, was placed under control of the P_LtetO-1 promoter in a second neutral locus denoted Ntrl2, replacing the gene remnants ACIAD1076-1077. Repeated attempts to clone wild type tetR into A. baylyi failed, but a temperature sensitive mutant was isolated with two mutations: W75R and an additional 8 amino acids on the C terminus. This mutant tetR permitted growth at both 30° C. and 37° C., but it only repressed its target at 30° C. Wild-type tetR was cloned on the inducer aTc, but it was unable to grow without aTc at any temperature.

### In Vitro Biosensor Transformation Experiments

A. baylyi were grown overnight in LB at 30° C. Cells were then washed, resuspended in an equal volume of fresh LB, and mixed with donor DNA. For transformation in liquid, 50 μl cells were mixed with 250 ng donor DNA and incubated in a shaker at 30° C. for 2 hours or overnight. For transformation on agar, 2 μl cells were mixed with >50 ng donor DNA, spotted onto LB plates containing 2% wt/vol agar, and incubated at 30° C. overnight. Spots were cut out the next day and resuspended in 500 μl phosphate buffered saline solution (PBS). To count transformants, cells were 10-fold serially diluted 5 times, and 2 μl spots were deposited onto selective (30 ng/mL kanamycin) and non-selective 2% agar plates, with 3 measurement replicates at each dilution level. Larger volumes of undiluted samples were also spread onto agar plates to increase detection sensitivity (25 μl for liquid culture, 100 μl for resuspended agar spots). Colonies were counted at the lowest countable dilution level after overnight growth at 30° C., and measurement replicates were averaged. Raw, unpurified lysate was produced by growing donor RKO cells in a culture dish until confluence, trypsinizing and harvesting cells, pelleting them in a 15 mL tube, resuspending them in 50 μl PBS, and placing the tube in a −20° C. freezer overnight to disrupt cell membranes.

### In Vitro Statistics

Hypothesis testing was performed using two sample, one-sided t-tests in Matlab after taking base 10 logarithms, since serial dilutions produce log-scale data. Where data points were below the limit of detection, they were replaced by the limit of detection as the most conservative way to include them in log-scale analysis. Comparisons between large vs small inserts or liquid vs solid agar culture were performed using paired t-tests, where data were matched for donor DNA and either culture type (liquid vs agar) or insert size, respectively. For FIGS. 2D-2G: n=4, FIGS. 2I-2J: n=5 except for random spacer n=3.

### Creation of RKO, LS174T and SW620 Donor Cell Lines

To create RKO donor and LS174T donor cell lines, lentiviral expression plasmid pD2119-FLuc2 KRasG12D donor was co-transfected with viral packaging vectors, psPAX2 (Addgene; plasmid; 12260) and MD2G (Addgene; plasmid; 12259), into HEK293T cells. At 48 hours and 72 hours after transfection, viral supernatants were harvested, filtered through a 0.45-μm filter, and concentrated using Amicon Ultra Centrifugal Filters (Merck Millipore; UFC910024). Concentrated lentivirus particles were used for transduction. The viral supernatant generated was used to transduce RKO, LS174T and SW620 cells. 48 hours of transduction, stable transformants were selected with 4 μg/mL puromycin. Cell line identity was confirmed by STR analysis. KRAS status of RKO (KRAS wildtype), LS174T (KRAS G12D) and SW620 (KRAS G12V) cell lines was confirmed by amplification of a 220 bp PCR fragment of the exon 2 KRAS gene, including codons 12 and 13 with primers KRAS F: GGTGGAGTATTTGATAGTGTATTAACC (SEQ ID NO: 1) and KRAS R: AGAATGGTCCTGCACCAGTAA (SEQ ID NO: 2). Sanger sequencing was conducted using the same primers.

### Creation of CRC Donor Organoids

BTRZI (BrafV/600E; Tgfbr24 4;Rnf43Δ/Δ; Znf43Δ/Δ; p16 Ink4a Δ/Δ) organoids were generated using CRISPR-Cas9 engineering and grown in 50 μl domes of GFR-Matrigel (Corning; 356231) in organoid medium: Advanced Dulbecco's modified Eagle medium/F12 (Gibco; 12634010) supplemented with 1× gentamicin/antimycotic/antibiotic (Gibco; 15710064, 15240062), 10 mM HEPES (Gibco; 15630080), 2 mM GlutaMAX (Gibco; 35050061), 1×B27 (Gibco; 12504-044), 1×N2 (Gibco; 17502048), 50 ng/mL mouse recombinant EGF (Peprotech; 315-09), 10 ng/mL human recombinant TGF-β1 (Peprotech; 100-21) (26). Following each split, organoids were cultured in 10 μM Y-27632 (MedChemExpress; HY-10583), 3 μM iPSC (Calbiochem; 420220), 3 μM GSK-3 inhibitor (XVI, Calbiochem; 361559) for the first 3 days. Human colorectal tumor samples were obtained at the time of surgery for routine pathological analysis. All participants gave written informed consent before the research was conducted in accordance with the Declaration of Helsinki, the NHMRC Statement on Ethical Conduct. Tumor organoid lines were derived as described elsewhere (Sato et al., Gastroentr. 141:1762-1772 (2011)), with minor modifications. Tissue samples were first minced and enzymatically digested in organoid digestion medium containing advanced DMEM-F12 (Gibco; 12634-010), Antibiotic-Antimycotic (Gibco; 15240-062), 50 μg/mL Gentamicin (Gibco; 15750-060), 1% FCS, Collagenase IV 75 U/mL (Gibco; 17104019), Dispase 125 μg/mL (Gibco; 17105-041), Hyaluronidase 20 μg/mL (Sigma; H3506), Y27632 (Sigma; Y0503) in a water bath at 37° C. for 10 minutes then a further 30 minutes shaking at 150 rpm in an orbital shaker at 37° C. Samples were strained, washed and plated in domes of GFR-Matrigel (Corning; 356231). Human CRC organoids were cultured in colorectal cancer medium containing advanced DMEM/F12 (Gibco; 12634-010), Antibiotic-Antimycotic (Gibco; 15240-062), 50 μg/mL Gentamicin (Gibco; 15750-060), 10 mM HEPES (Sigma; H0887), 1×glutamax (Gibco; 35050-061), 2×B27 (Gibco; 17504044), 500 nM A83-01 (Tocris; 2939), 50 ng/ml hEGF (Sigma; SRP3027), 1 nM [Leu15]-Gastrin 1 (Sigma; G9145), 1 mM N-Acetyl-Lcysteine (Sigma; A9165), 5 μM SB202190 (Sigma; S7067), 10 μM SB431542 (Sigma; S4317), and 10 μM Y27632 (Sigma; Y0503). Colorectal cancer medium was changed twice weekly, with growth monitored until passaging was required.

To create CRC donor organoids, lentiviral expression plasmid pD2119-FLuc2 KRASG12D donor was co-transfected with viral packaging vectors, psPAX2 (Addgene; plasmid; 12260) and MD2G (Addgene; plasmid; 12259), into HEK293T cells. At 48 hours and 72 hours after transfection, viral supernatants were harvested, filtered through a 0.45-μm filter, and concentrated using Amicon Ultra Centrifugal Filters (Merck Millipore; UFC910024). Concentrated lentivirus particles were used for transduction. The viral supernatant generated was used to transduce human CRC and BTRZI organoids by spinoculation. Briefly, organoids were dissociated to single cells using TrypLE. 1×10⁵ single cells were mixed with 250 μl organoid medium; 10 μM Y-27632; 250 μl concentrated viral supernatant and 4 μg/mL polybrene (Sigma; H9268) in a 48 well tray before centrifugation at 600 g for 90 minutes at 32° C. Meanwhile, 120 μl 50:50 ADMEM: Matrigel mixture was added to a cold 24-well tray before centrifugation of this bottom Matrigel layer for 40 minutes at 200 g at room temperature, followed by solidifying the Matrigel by incubating at 37° C. for 30 minutes. After spinoculation, cells were scraped from the well and plated on top of the Matrigel monolayer with organoid medium. The following day, the medium was removed and the upper layer of Matrigel was set over the organoids by adding 120 μl 50:50 ADMEM: Matrigel and allowing to set for 30 minutes before adding organoid medium. 48 hours after transduction, BTRZI donor organoids were selected with 8 μg/mL puromycin (Sigma; P8833) for 1 week, then maintained in organoid medium with 4 μg/mL puromycin. Human CRC donor organoids were selected and maintained in 4 μg/mL puromycin.

Calculating number of copies of target DNA integrated into cell lines and organoids Genomic DNA (gDNA) was extracted from 5×10⁶ cells using Purelink genomic DNA minikit (Invitrogen; K18200). Primer/probe sets were designed to amplify the human KRAS homology arms within the donor construct. 5′KRAS homology Arm FWD 5′-CAG AAC AGC AGT CTG GCT ATT TA-3′ (SEQ ID NO: 3); 5′KRAS homologyArm REV 5′-ACT GCA GAC GTG TAT CGT AAT G-3′ (SEQ ID NO: 4) and 5′ KRAS homology Arm PRB 5′-/56-FAM/AGC GTC GAT/ZEN/GGA GGA GTT TGT AAA TGA/31ABKFQ/-3′ (SEQ ID NO: 5). Quantitative PCR (qPCR) reactions were optimized with normal human gDNA (from PBMC), mouse gDNA (BTRZI parental organoid gDNA) and BTRZI donor gDNA to show that primer/probe sets amplify both endogenous human KRAS and the donor construct, but not mouse Kras. To enable calculation of copy number of each cell and organoid line, a standard curve was generated with normal human gDNA (2-fold serial dilution 100 ng to 3.125 ng per qPCR reaction). qPCR was conducted on 25 ng gDNA from organoids and cell lines. All qPCR reactions were normalized to 100 ng per qPCR reaction with mouse gDNA (BTRZI parental organoid gDNA) and used KAPA probe fast universal (Roche; KK4703).

Organoid Lysate Mixed with A. baylyi Sensor Bacteria

BTRZI (parental) and BTRZI donor organoids were grown for 5 days in 50 μl GFR-Matrigel domes. Organoids were dissociated to single cells with TrypLE, counted and 6×105 single cells were collected in PBS and snap frozen. The CFU equivalence of exponentially growing A. baylyi sensor culture at OD600 0.35 was ascertained by serial dilution of 3 independent cultures with 5 technical replicates plated on 10 μg/mL chloramphenicol LB agar plate to be 2.4×108 CFU per mL. A. baylyi sensor was grown in liquid culture with 10 μg/mL chloramphenicol to OD600 0.35 before mixing with organoid lysate at a 1:1 ratio and grow overnight on LB agar plates at 30° C. All bacteria were scraped into 200 μl LB/20% glycerol before spotting 5×5 μl spots onto kanamycin (Sigma; K1377) and chloramphenicol (Sigma; C0378) plates and grown overnight at 37° C. Colonies were counted and the dilution factor was accounted for to calculate CFU per mL. Rate of HGT was calculated by dividing the CFU per mL of transformants (kanamycin plates) by the CFU per of total A. baylyi (chloramphenicol plates) for 5 independent experiments.

Coculture Organoids with A. baylyi Sensor Bacteria

For co-culture experiments, 24-well trays were coated with Matrigel monolayers. Briefly, 200 μl 50:50 ADMEM: Matrigel mixture was added to a cold 24-well tray and centrifuged for 40 minutes at 200×g at room temperature, followed by a 30-minute incubation at 37° C. to solidify Matrigel. BTRZI (parental) and BTRZI donor organoids were dissociated into small clusters using TrypLE and grown for 5 days on a Matrigel monolayer in organoid medium without antibiotics before 50 μl OD600 0.35 A. baylyi sensor was added to each well. After 24 hours, organoids were photographed then collected and grown overnight on LB agar plates at 30° C. All bacteria were scraped into 200 μl LB/20% glycerol before spotting 5×5 μl spots onto kanamycin and chloramphenicol plates and grown overnight at 37° C. Colonies were counted and the dilution factor was accounted for to calculate CFU per mL. Rate of HGT was calculated by dividing the CFU per mL of transformants (kanamycin plates) by the CFU per mL of total A. baylyi (chloramphenicol plates) for 5 independent experiments.

### Horizontal Gene Transfer In Vivo

BTRZI donor organoids were isolated from Matrigel and dissociated into small clusters using TrypLE. The cell clusters (equivalent to ˜150 organoids per injection) were washed three times with cold PBS containing 10 μM Y-27632 and then resuspended in 20 μl 10% GFR Matrigel 1:1000 Indian ink, 10 μM Y-27632 in PBS and orthotopically injected into the mucosa of the proximal and distal colon of anaesthetized 10- to 13-week-old NSG mice (150 organoids per injection). Briefly, a customized needle (Hamilton Inc. part number 7803-05, removable needle, 33 gauge, 12 inches long, point 4, 12° bevel) was used. In each mouse up to 2 injections of 20 μl were performed. CRC donor tumor growth was monitored by colonoscopy for 5 weeks and the videos were viewed offline using QuickTime Player for analysis. Colonoscopy was performed using a Karl Storz Image 1 Camera System comprised of: Image 1 HDTV HUB CCU; Cold Light Fountain LED Nova 150 light source; Full HD Image 1 3 Chip H3-Z Camera Head; Hopkins Telescope, 1.9 mm, 0 degrees. A sealed Luer Lock was placed on the working channel of the telescope sheath to ensure minimal air leakage (Coherent Scientific; 14034-40). Tumor growth of the largest tumor visualized was scored using the Becker Scale as described elsewhere (Becker et al., Nat. Protoc. 1:2900-2904 (2006)). All study groups were housed in separate cages. A. baylyi sensor was grown in LB liquid culture with 6 μg/mL chloramphenicol to OD600 0.6. A. baylyi parental was grown in LB liquid culture to OD600 0.6. A. baylyi was washed twice with PBS before 13 mice received 4×1010 A. baylyi sensor via enema (5 mice without tumors; 3 mice with non-donor BTRZI CRC tumors and 5 mice with BTRZI CRC donor tumors), 4 mice received 4×1010 A. baylyi parental via enema. Briefly, mice were anesthetized with isoflurane and colon was flushed with 1 mL of room temperature sterile PBS to clear the colon cavity of any remaining stool. A P200 pipette tip coated with warm water was then inserted parallel into the lumen to deliver 50 μl of bacteria into the colon over the course of 30 seconds. After infusion, the anal verge was sealed with Vetbond Tissue Adhesive (3M; 1469SB) to prevent luminal contents from being immediately excreted. Animals were maintained on anesthesia for 5 minutes, and then allowed to recover on a heat mat and anal canal was inspected 6 hours after the procedure to make sure that the adhesive has been degraded. 24 hours after A. baylyi administration, mice received a second enema dosing. Mice were then euthanized; colons were removed, and luminal contents were collected. Luminal contents were grown overnight at 37° C. on LB agar with 10 μg/mL vancomycin plates. All bacteria were collected into 250 μl LB/20% glycerol, vortexed and stored at −80° C. Serial dilutions of 5×5 μl were spotted onto LB agar plates containing: 1) vancomycin (to detect total A. baylyi parental); 2) chloramphenicol; vancomycin (to detect total A. baylyi sensor) and 3) kanamycin; chloramphenicol; vancomycin (to detect recombined A. baylyi sensor). Colonies were counted and dilutions were factored to calculate CFU A. baylyi per mouse. For experiments analyzing A. baylyi in stool, BTRZI CRC donor tumors were established and monitored as described above. After 5 weeks of tumor growth, 9 mice received A. baylyi sensor enemas (5 mice without tumors; 4 mice with BTRZI CRC donor tumors) and 6 mice received A. baylyi parental enemas (3 mice without tumors and 3 mice with BTRZI CRC donor tumors). Stool was collected 24 hours after A. baylyi administration into 250 μl PBS/20% glycerol, vortexed and stored at −80° C. Stool was analyzed on LB agar plates containing: 1) vancomycin (Sigma; PHR1732) (to detect total A. baylyi parental); 2) chloramphenicol; vancomycin (to detect total A. baylyi sensor) and 3) kanamycin; chloramphenicol; vancomycin (to detect recombined A. baylyi sensor). Colonies were counted and dilutions were factored to calculate CFU A. baylyi per mouse.

Sequencing gDNA from Bacterial Colonies Grown on Kanamycin Plates

A. baylyi transformants were individually picked from kanamycin; vancomycin plates and grown in liquid culture LB supplemented with 25 μg/mL kanamycin, 10 μg/mL vancomycin and 6 μg/mL chloramphenicol. gDNA was extracted using purelink genomic DNA minikit (Invitrogen; K182001). Genomic regions of interest were amplified using Primestar Max DNA polymerase (Takara; R045A) and primers HGTpcrF: CAAAATCGGCTCCGTCGATACTA (SEQ ID NO: 6); HGTpcrR: TAGCATCACCTTCACCCTC (SEQ ID NO: 7) and 16S 27Fa: AGAGTTTGATCATGGCTCAG (SEQ ID NO: 8); 16s 27Fc: AGAGTTTGATCCTGGCTCAG (SEQ ID NO: 9); 16S 1492R: CGGTTACCTTGTTACGACTT (SEQ ID NO: 10) (16S 27Fa: 16S 27Fc: 16S 1492R=0.5:0.5:1). Sanger sequencing was conducted using the same primers.

### Calculating the Detection Limit of CATCH in the Presence of Stool

Stools were collected from C57BL/6 mice bearing CRC tumors. A stool slurry was created by vortexing 10 mouse stools (average weight 0.017 g) in 830 μl LB/20% glycerol for each experiment, then 100 μl stool slurry could be added to each reaction containing the equivalent of 1 stool. Donor plasmid DNA was serially diluted (10-fold from 3 ng/μl to 30 fg/μl). A. baylyi sensor was grown in LB liquid culture with 6 μg/mL chloramphenicol to OD600 0.6. 5×107 A. baylyi sensor was mixed with 100 μl stool slurry or 100 μl LB/20% glycerol with or without serially diluted donor plasmid DNA. Reactions were incubated at 37° C. for 4 hours before serially diluting (10-fold) and spotting 5×5 μl spots onto 25 μg/mL kanamycin; 10 μg/mL vancomycin LB agar plates and 6 μg/mL chloramphenicol; 10 μg/mL vancomycin LB agar plates and grown overnight at 37° C. Colonies were counted and the dilution factor was accounted for to calculate CFU per mL. Rate of HGT was calculated by dividing the CFU per mL of transformants (kanamycin plates) by the CFU per mL of total A. baylyi (chloramphenicol plates) for 5 independent experiments.

### Example 2: Use of Biosensors in Other Samples

Biosensors can be used in other samples, for example, urine, sputum, and blood samples, for which other detection techniques require prior purification. The biosensors described in the present disclosure can be used to diagnose hard-to-culture uropathogens, identify antibiotic resistant mutations, or subtype E. coli for public health monitoring.

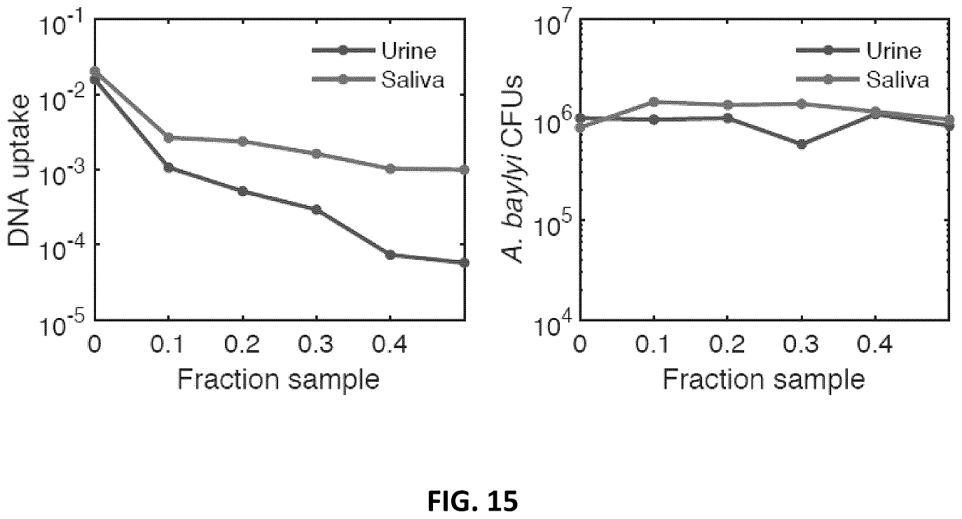

After incubating A. baylyi with known donor DNA mixed with human samples such as urine and saliva, A. baylyi still retains its natural competence (FIG. 15), and its survival is not impeded by concentrations of at least 50% (FIG. 15). DNA degradation rate in the sample is tested by mixing known concentrations of DNA with urine and varying concentrations of actin or anti-DNase, extracting and purifying DNA at several time points, and measuring the DNA concentration with a qubit or qPCR. Appropriate concentrations of inhibitors are verified in full biosensing reactions.

To test the effects of A. baylyi biosensor strains on uropathogen detection, the urine sample is tested for Actinotignum schaalii (formerly Actinobaculum). Target homology regions in the genome are identified with maximal Hamming distance from other DNA sequences in Genbank. For CRISPR-based and single-base discrimination step, CRISPR targets that maximally differentiate the intended DNA from the closest potential off-target matches are identified.

To test the effects of A. baylyi biosensor strains on antibiotic resistant mutations, the study focuses on mutations of a single nucleotide in DNA gyrase A (gyrA), that is found to be responsible for roughly 75% of resistance to nalidixic acid and fluoroquinolones in Salmonella enterica Typhi and Paratyphi. For E. coli subtyping, the study tests for 2 single nucleotide polymorphisms (SNPs) within the icdA gene that largely distinguishes between phylotypes B2 and D, which are more commonly pathogenic, vs A and B1, which are most commonly commensal. In both cases, A. baylyi biosensor strains are constructed with the appropriate homology arms and CRISPR spacers targeting the relevant SNPs, which are in the A. baylyi PAM sequence. Additionally, attenuated Salmonella and E. coli Nissle strains are constructed with identical target and control strains for detection of antibiotic resistant mutations and E. coli subtyping, respectively. E. coli subtyping uses two biosensor strains, one that produces output for each subtype. In this condition, one biosensor strain can be used to detect pathogenic E. coli, and one to detect nonpathogenic E. coli in the same sample.

Biosensing of target DNA is validated using PCR amplicons, purified genomic DNA, and raw bacterial cultures. The target DNA is quantified, and the output is determined using kanamycin resistance and lateral flow assay. After validation in vitro, the Actinotignum detection and E. coli subtyping biosensors are tested in urine. Urine samples from 10 individuals are spiked with known Actinotignum and E. coli cells at a range of concentrations. The urine samples are incubated for 1 hour at 37° C., and then used as samples for biosensing. Additionally, biosensing is tested for all the targets and DNA sources in other samples such as blood and sputum. A. baylyi directly lyses other bacteria, including E. coli, rendering their DNA available for uptake for homologous recombination. Gram-positive bacteria can release DNA by adding lysozyme to the incubation buffer. Gram negative bacteria can release DNA by adding partially denatured lysozyme. To protect A. baylyi from lysozyme, A. baylyi biosensor bacteria are engineered to overexpress ivy, the gene that protects against lysozyme. To lyse Gram-positive and Gram-negative bacteria, both native and partially heat denatured lysozyme are added, respectively. To determine the impact of DNA sequestration inside cells, sterilized samples are inoculated with target strains, and incubated with or without A. baylyi and lysozymes. The fraction of free DNA vs intact cells for these spiked samples as well as direct clinical samples are determined. Samples are centrifuged to pellet intact cells, and then qPCR is performed on both the cell-free supernatant and the extracted genomic DNA from the pellet. Furthermore, these samples and samples containing equal amounts of free DNA are used for biosensing assays.

To test the detection of microbial DNA directly from unmodified clinical samples, urine samples are collected from Urinary Tract Infection (UTI) patients. Samples provided are deidentified blinded diagnostic status, but includes at least 10 samples for which expanded quantitative urine culture reveals the presence of Actinotignum or E. coli. Target DNA concentrations are confirmed via qPCR or droplet digital PCR, and the total DNA concentrations are quantified with a qubit. Sensitivity, specificity, and limits of detection are determined.

### Example 3: Target-Sensing Biosensor with Lateral Flow Assay Output

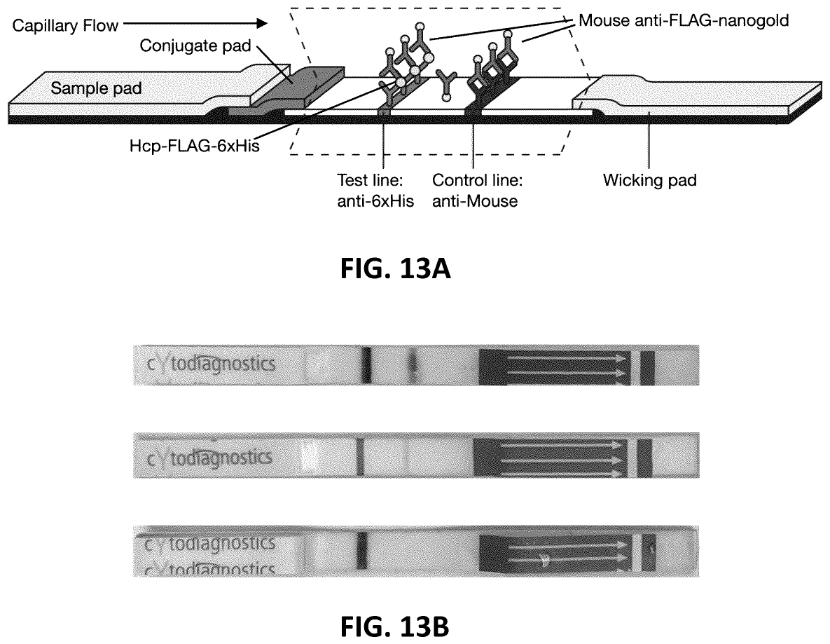

The engineered biosensor circuit is modular, and therefore, the output can be anything that is genetically encoded (FIGS. 4A-4E). To test if the output signaling molecule can be secreted into the media for detection on a lateral flow strip, a universal lateral flow assay kit (Cytodiagnosites Inc, Canada) is used, which requires two antibodies that each bind the signaling molecule (FIGS. 13A-13B). Lateral flow device is a user-friendly, point-of-care diagnostic with minimal user interaction, no sample purification, and no equipment. Biotinylated anti-His is used as a capture antibody, which also binds pre-immobilized streptavidin at the test line. FITC-conjugated anti-FLAG is used as a detection antibody, which binds gold nanoparticles (40 nm) conjugated to anti-FITC. Hep is used as a signaling molecule because it is an integral component of the A. baylyi Type VI Secretion System (T6SS) and is naturally secreted at a rate of around 400 copies per minute per cell. FLAG epitope tags and 6×His are placed on the Hep molecule at its natural genomic locus, enabling specific detection by the corresponding capture antibody and the detection antibody. To prevent the biological samples from interfering with the biotin-streptavidin interaction of the kit, the strip can be first dissembled and the biotinylated anti-His antibody can be pre-applied directly to the location of the streptavidin test line. Mouse anti-FLAG is directly conjugated to reporter gold nanoparticles.

To isolate the lateral flow assay optimization from DNA biosensing, the lateral flow assay is performed using a known concentration of purified tagged Hep diluted into the sample (e.g., urine, saliva, or blood) to various concentrations. After running the assay, the test lines are imaged, and their intensities are quantified. The combination of variables producing the strongest intensity band at the lowest concentration of Hcp (lowest limit of detection) are selected for further use, possibly with different formulations for different sample types. In addition to optimizing the physical assay components, the sensitivity is improved by increasing the number of gold particles bound by each secreted Hcp; i.e., increasing its valency. Additional FLAG tags are placed at the N terminus and at unstructured loops in the crystal structure. Each FLAG tag position is tested separately in the Hcp-producing strains grown to saturation, along with the 6×His tags at the C-terminus, to ensure that it does not diminish secretion and is compatible with detection on the lateral flow strip. Multivalent versions are also tested. While the number of antibody-conjugated gold nanoparticles that can be bound to an Hep hexamer may be limited by steric hindrance, additional FLAG tags can increase the band intensity 2- to 4-fold for a given concentration of Hcp.

The lateral flow biosensors are tested using various DNA sources and sample substrates. The secreted dual-tagged Hcp is quantified by Enzyme Linked Immunosorbent Assay (ELISA) as well as lateral flow assay, to separately calculate the levels of Hep secretion and the lateral flow biosensing step. Imaging processing software (e.g., Image J and Matlab) or applications are used to quantify band intensity.

To facilitate low-cost storage and delivery without requiring a cold chain, the biosensors are dried in a test vial, to which samples and incubation buffer are added to perform an assay. Dry storage is tested for A. baylyi biosensors using vacuum drying and lyophilization. Sucrose, Ficoll, glycine betaine, and choline are used as protectants in A. baylyi or other species. The protectants are first tested to ensure that they do not reduce DNA uptake. Dried cells are rehydrated with media containing target DNA. The total survival, DNA sensing, and signal molecule secretion are compared to a culture of fresh cells. The lowest target concentration that produces a positive result is the limit of detection. For unmodified clinical samples, a portion of the sample is used to extract and purify total DNA, from which the true target concentration is determined using a qPCR or a droplet digital PCR and multiplied by the dilution factor. The intensity of the test band on the strip is measured as the output on the lateral flow device. To quantify the output, the band intensity is calculated using an adjacent standard curve for normalization. Assays is considered positive when they produce a visible test line.

### Example 4: Target-Sensing Biosensor with CRISPR-Associated Transposons (CAST) Output

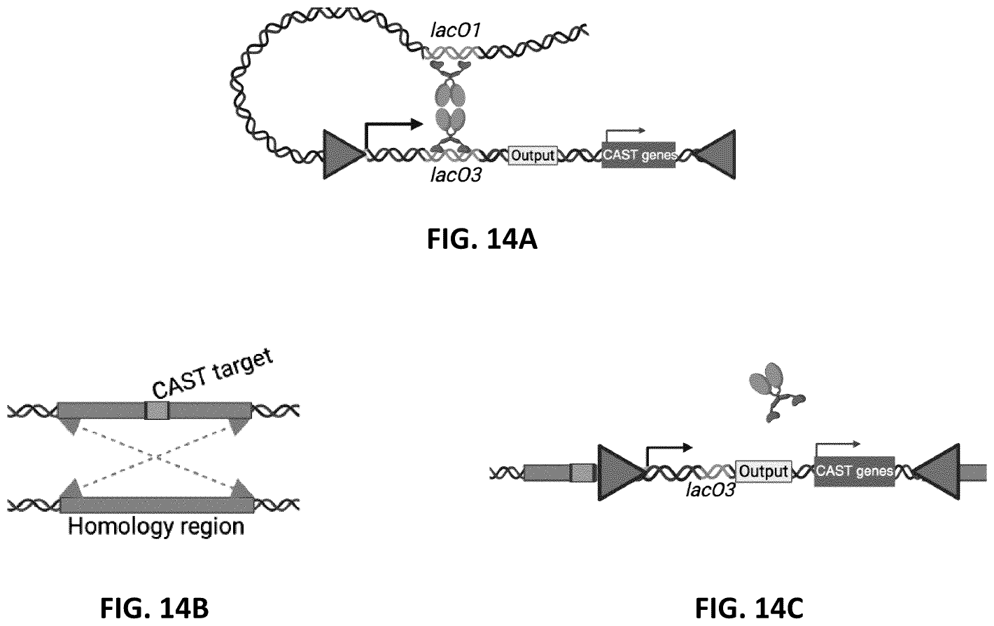

The CRISPR-associated transposon (CAST) system uses DNA looping in the lac operon. In this approach, the homology region in the A. baylyi genome is fully matched with the target DNA, except for a small target region that is the size of a CRISPR target site and its PAM site. The second locus has a CRISPR-guided, site-specific transposon. This family of CRISPR-associated transposons or CASTs jump only to a sequence-specific target site, defined by their CRISPR guide RNAs. This transposon carries an output gene as a cargo, but its expression is repressed by DNA looping with a repressor binding site just outside the transposon (FIG. 14). The CAST is targeted to a small 30 bp target sequence by placing the cognate spacer into its CRISPR array. Uptake and recombination with the target DNA sequence introduces the CAST target (FIG. 14B), thus allowing the transposon to recognize and jump into its programmed target, and away from DNA looping, allowing the expression of its output cargo (FIG. 14C).

When the lac operators are rearranged such that a lone, relatively poor LacI binding site (lacO3) is placed directly downstream of the promoter, there is no repression. However, when a very strong binding site (lacO1) is added around 100 bp upstream (in periodic intervals for proper physical orientation on the same side of the double helix), repression increases to around 1000-fold due to looping. The ideal spacing between lacO sites is around the length of transposon end sequences, which allows placement of the promoter and the weak lacO3 inside the transposon, and the strong lacO1 outside. Thus, the output gene is tightly repressed initially, but is nearly completely derepressed after transposition to a programmed target site following recombination with the target DNA.

The output gene is placed under the control of lacO sites, with the weak lacO3 inside the CAST transposon and the strong lacO1 outside. The CAST is targeted to a sequence-specific site in the target DNA using a CAST guide RNA. Guide RNAs are designed to have maximal Hamming distance from all other sites on the A. baylyi genome, thus preventing off-site transposition. The separate homologous recombination site has 2 kb of complete homology to the target, except for the region targeted by the CAST guide RNA. Since sensing does not depend on the deletion of a single repressor gene in this study, LacI is expressed from two independent regions on the genome, thus decreasing the rate of false positive result due to repressor mutations. This design is tested using the leading CASTs from V. cholera, A. salmonicida, and other promising CAST candidates. DNA sensing sensitivity, false positive rate, and tightness of repression are quantified. Any false positives growing on selection plates without having been exposed to the target DNA are sequenced to determine the failure mode.

### REFERENCES

1. S. Slomovic, K. Pardee, J. J. Collins, Proc. Natl. Acad. Sci. U.S.A. 112, 14429-14435 (2015).
2. F. Sedlmayer, D. Aubel, M. Fussenegger, Nat. Biomed. Eng. 2, 399-415 (2018).
3. W. A. Lim, C. H. June, Cell 168, 724-740 (2017).
4. D. T. Riglar et al., Nat. Biotechnol. 35, 653-658 (2017).
5. M. Mimee et al., Science 360, 915-918 (2018).
6. N. Mao, A. Cubillos-Ruiz, D. E. Cameron, J. J. Collins, Sci. Transl. Med. 10, caao2586 (2018).
7. T. Danino et al., Sci. Transl. Med. 7, 289ra84 (2015).
8. J. W. Kotula et al., Proc. Natl. Acad. Sci. U.S.A. 111, 4838-4843 (2014).
9. T. S. Gardner, C. R. Cantor, J. J. Collins, Nature 403, 339-342 (2000).
10. A. Courbet, D. Endy, E. Renard, F. Molina, J. Bonnet, Sci. Transl. Med. 7, 289ra83 (2015).
11. J. C. Mell, R. J. Redfield, J. Bacteriol. 196, 1471-1483 (2014).
12. S. M. Soucy, J. Huang, J. P. Gogarten, Nat. Rev. Genet. 16, 472-482 (2015).
13. K. M. Robinson, K. B. Sieber, J. C. Dunning Hotopp, PLOS Genet. 9, e1003877 (2013).
14. J. C. Dunning Hotopp, Trends Genet. 27, 157-163 (2011).
15. D. M. Young, D. Parke, L. N. Ornston, Annu. Rev. Microbiol. 59, 519-551 (2005).
16. R. Palmen, B. Vosman, P. Buijsman, C. K. D. Breck, K. J. Hellingwerf, J. Gen. Microbiol. 139, 295-305 (1993).
17. T.-L. Chen et al., J. Clin. Microbiol. 46, 2938-2944 (2008).
18. L. Nordgård et al., Environ. Biosafety Res. 6, 149-160 (2007).
19. R. M. Cooper, L. Tsimring, J. Hasty, eLife 6, e25950 (2017).
20. B. Vogelstein et al., N. Engl. J. Med. 319, 525-532 (1988).
21. D. J. Simpson, L. F. Dawson, J. C. Fry, H. J. Rogers, M. J. Day, Environ. Biosafety Res. 6, 55-69 (2007).
22. F. André et al., Cancer Discov. 7, 818-831 (2017).
23. W. Li et al., Int. J. Clin. Exp. Pathol. 12, 957-967 (2019).
24. P. Priestley et al., Nature 575, 210-216 (2019).
25. R. M. Cooper, J. Hasty, ACS Synth. Biol. 9, 1129-1137 (2020).
26. T. R. M. Lannagan et al., Gut 68, 684-692 (2019).
27. R. Lutz, H. Bujard, Nucleic Acids Res. 25, 1203-1210 (1997).
28. C. Myhrvold et al., Science 360, 444-448 (2018).
29. J. S. Chen et al., Science 360, 436-439 (2018).
30. Y. Zhong, F. Xu, J. Wu, J. Schubert, M. M. Li, Ann. Lab. Med. 41, 25-43 (2021).
31. M. Iwamoto et al., MMWR Morb. Mortal. Wkly. Rep. 64, 252-257 (2015).
32. O. Shimada et al., J. Histochem. Cytochem. 46, n833-840 (1998).
33. A. Wilcks, A. H. A. M. van Hock, R. G. Joosten, B. B. L. Jacobsen, H. J. M. Aarts, Food Chem. Toxicol. 42, 493-502 (2004).
34. T. Netherwood et al., Nat. Biotechnol. 22, 204-209 (2004).
35. M. O. Din et al., Nature 536, 81-85 (2016).
36. G. D. Sepich-Poore et al., Science 371, cabc4552 (2021).
37. Jason Doyle and Sven Uthicke. Sensitive environmental DNA detection via lateral flow assay (dip-stick)—A case study on corallivorous crown-of-thorns sea star (Acanthaster cf. solaris) detection. En-vironmental DNA, 3 (2): 323-342, 2021. doi: 10.1002/edn3.123.
38. Baylie R. Hochstedler, Lindsey Burnett, Travis K. Price, Carrie Jung, Alan J. Wolfe, and Linda Brubaker. Urinary microbiota of women with recurrent urinary tract infection: collection and culture methods. International Urogynecology Journal, 33 (3): 563-570, 2022. doi: 10.1007/s00192-021-04780-4.
39. Antti Vasala, Vesa P. Hytönen, and Olli H. Laitinen. Modern Tools for Rapid Diagnostics of Antimicrobial Resistance. Frontiers in Cellular and Infection Microbiology, 10:308, 2020. doi: 10.3389/fcimb.2020.00308.
40. Alex van Belkum, Till T Bachmann, Gerd Lüdke, Jan Gorm Lisby, Gunnar Kahlmeter, Allan Mohess, Karsten Becker, John P Hays, Neil Woodford, Konstantinos Mitsakakis, Jacob Moran-Gilad, Jordi Vila, Harald Peter, John H Rex, Wm Michael Dunne, and Testing, JPIAMR AMR-RDT Working Group on Antimicrobial Resistance and Rapid Diagnostic. Developmental roadmap for antimicrobial susceptibility testing systems. Nature Reviews Microbiology, 17 (1): 51-62, 2019. doi: 10.1038/s41579-018-0098-9.
41. Kevin J. Land, Debrah I. Boeras, Xiang-Sheng Chen, Andrew R. Ramsay, and Rosanna W. Peeling. REASSURED diagnostics to inform disease control strategies, strengthen health systems and improve patient outcomes. Nature Microbiology, 4 (1): 46-54, 2019. doi: 10.1038/s41564-018-0295-3.
42. Jane Y. Zhang, Andrew T. Bender, David S. Boyle, Paul K. Drain, and Jonathan D. Posner. Current state of commercial point-of-care nucleic acid tests for infectious diseases. Analyst, 146 (8): 2449-2462, 2021. doi: 10.1039/d0an01988 g.
43. Michael M. Kaminski, Omar O. Abudayyeh, Jonathan S. Gootenberg, Feng 43. Zhang, and James J. Collins. CRISPR-based diagnostics. Nature Biomedical Engineering, 5 (7): 643-656, 2021. doi: 10.1038/s41551-021-00760-7.
44. Martha Iwamoto, Jennifer Y Huang, Alicia B Cronquist, Carlota Medus, Sharon Hurd, Shelley Zansky, John Dunn, Amy M Woron, Nadine Oosmanally, Patricia M Griffin, John Besser, Olga L Henao, and (CDC), Centers for Disease Control and Prevention. Bacterial enteric infections detected by culture-independent diagnostic tests-FoodNet, United States, 2012-2014. MMWR. Morbidity and mortality weekly report, 64 (9): 252-7, 2015.
45. Shari Shea, Kristy A Kubota, Hugh Maguire, Stephen Gladbach, Amy Woron, Robyn Atkinson-Dunn, Marc Roger Couturier, Melissa B Miller, Shari Shea, Kristy A Kubota, Hugh Maguire, Stephen Glad-bach, Amy Woron, Marc Roger Couturier, Melissa B Miller, and Peter H Gilligan. Clinical Micro-biology Laboratories' Adoption of Culture-Independent Diagnostic Tests Is a Threat to Foodborne-Disease Surveillance in the United States. Journal of Clinical Microbiology, 55 (1): 10-19, 2017. doi: 10.1128/jcm.01624-16.
46. Aamer Imdad, Fiona Retzer, Linda S Thomas, Marcy McMillian, Katie Garman, Peter F Rebeiro, Stephen A Deppen, John R Dunn, and Amy M Woron. Impact of Culture-Independent Diagnostic Testing on Recovery of Enteric Bacterial Infections. Clinical Infectious Diseases, 66 (12): 1892-1898, 2017. doi: 10.1093/cid/cix1128.
47. Betsy Foxman. Epidemiology of urinary tract infections: incidence, morbidity, and economic costs. The American Journal of Medicine, 113 (1): 5-13, 2002. doi: 10.1016/s0002-9343 (02) 01054-9.
48. Meghan M. Pearce, Evann E. Hilt, Amy B. Rosenfeld, Michael J. Zilliox, Krystal Thomas-White, Cyn-thia Fok, Stephanie Kliethermes, Paul C. Schreckenberger, Linda Brubaker, Xiaowu Gai, and Alan J. Wolfe. The Female Urinary Microbiome: a Comparison of Women with and without Urgency Urinary Incontinence. mBio, 5 (4): e01283-14, 2014. doi: 10.1128/mbio.01283-14.
49. Elizabeth R. Mueller, Alan J. Wolfe, and Linda Brubaker. Female urinary microbiota. Current Opinion in Urology, 27 (3): 282-286, 2017. doi: 10.1097/mou.0000000000000396.
50. Kirk J. Wojno, David Baunoch, Natalie Luke, Michael Opel, Howard Korman, Colleen Kelly, S. Mo-hammad A. Jafri, Patrick Keating, Dylan Hazelton, Stephany Hindu, Bridget Makhloouf, David Wen-zler, Mansour Sabry, Frank Burks, Miguel Penaranda, David E. Smith, Andrew Korman, and Larry Sirls. Multiplex PCR Based Urinary Tract Infection (UTI) Analysis Compared to Traditional Urine Cul-ture in Identifying Significant Pathogens in Symptomatic Patients. Urology, 136:119-126, 2020. doi: 10.1016/j.urology.2019.10.018.
51. Alec Szlachta-McGinn, K. Marie Douglass, Un Young Rebecca Chung, Nicholas James Jackson, J. Cur-tis Nickel, and A. Lenore Ackerman. Molecular Diagnostic Methods Versus Conventional Urine Cul-ture for Diagnosis and Treatment of Urinary Tract Infection: A Systematic Review and Meta-analysis. European Urology Open Science, 44:113-124, 2022. doi: 10.1016/j.euros.2022.08.009.
52. Austin G. Rottinghaus, Matthew B. Amrofell, and Tae Seok Moon. Biosensing in Smart Engineered Probiotics. Biotechnology Journal, 15 (10): 1900319, 2020. doi: 10.1002/biot.201900319.
53. David T. Riglar and Pamela A. Silver. Engineering bacteria for diagnostic and therapeutic applications. Nature Reviews Microbiology, 16 (4): 214-225, 2018. doi: 10.1038/nrmicro.2017.172.
54. Danielle B Pedrolli, Nathan V Ribeiro, Patrick N Squizato, Victor N de Jesus, Daniel A Cozetto, Rafael B Tuma, Amanda Gracindo, Mariana B Cesar, Paulo J C Freire, Ana F M da Costa, Milca R C R Lins, Graciely G Correa, and Marcel O Cerri. Engineering Microbial Living Therapeutics: The Synthetic Biology Toolbox. Trends in Biotechnology, 37 (1): 100-115, 2018. doi: 10.1016/j.tibtech.2018.09.005.
55. Niharika Gupta, Venkatesan Renugopalakrishnan, Dorian Liepmann, Ramasamy Paulmurugan, and Bansi D. Malhotra. Cell-based biosensors: Recent trends, challenges and future perspectives. Biosensors and Bioelectronics, 141:111435, 2019. doi: 10.1016/j.bios.2019.111435.
56. Minjeong Kang, Donghui Choe, Kangsan Kim, Byung-Kwan Cho, and Suhyung Cho. Synthetic Bi-ology Approaches in the Development of Engineered Therapeutic Microbes. International Journal of Molecular Sciences, 21 (22): 8744, 2020. doi: 10.3390/ijms21228744.
57. Maria Eugenia Inda and Timothy K. Lu. Microbes as Biosensors. Annual Review of Microbiology, 74 (1): 1-23, 2020. doi: 10.1146/annurev-micro-022620-081059.
58. Matthew B Amrofell, Austin G Rottinghaus, and Tae Seok Moon. Engineering microbial diagnos-tics and therapeutics with smart control. Current Opinion in Biotechnology, 66:11-17, 2020. doi: 10.1016/j.copbio.2020.05.006.
59. Garrett Graham, Nicholas Csicsery, Elizabeth Stasiowski, Gregoire Thouvenin, William H Mather, Michael Ferry, Scott Cookson, and Jeff Hasty. Genome-scale transcriptional dynamics and environ-mental biosensing. Proceedings of the National Academy of Sciences, 117 (6): 3301-3306, 02 2020. doi: 10.1073/pnas. 1913003117.
60. Alexis Courbet, Drew Endy, Eric Renard, Franck Molina, and Jerome Bonnet. Detection of patholog-ical biomarkers in human clinical samples via amplifying genetic switches and logic gates. Science Translational Medicine, 7 (289): 289ra83 289ra83, 05 2015. doi: 10.1126/scitranslmed.aaa3601.
61. David T Riglar, Tobias W Giessen, Michael Baym, S Jordan Kerns, Matthew J Niederhuber, Roderick T Bronson, Jonathan W Kotula, Georg K Gerber, Jeffrey C Way, and Pamela A Silver. Engineered bacteria can function in the mammalian gut long-term as live diagnostics of inflammation. Nature Biotechnology, 35 (7): 653-658, 2017. doi: 10.1038/nbt.3879.
62. Mark Mimee, Phillip Nadeau, Alison Hayward, Sean Carim, Sarah Flanagan, Logan Jerger, Joy Collins, Shane McDonnell, Richard Swartwout, Robert J. Citorik, Vladimir Bulovic', Robert Langer, Giovanni Traverso, Anantha P. Chandrakasan, and Timothy K. Lu. An ingestible bacterial-electronic system to monitor gastrointestinal health. Science, 360 (6391): 915-918, 2018. doi: 10.1126/science.aas9315.
63. Ning Mao, Andres Cubillos-Ruiz, D Ewen Cameron, and James J Collins. Probiotic strains detect and suppress cholera in mice. Science Translational Medicine, 10 (445): eaao2586, 06 2018. doi: 10.1126/sci-translmed.aao2586.
64. Tal Danino, Arthur Prindle, Gabriel A Kwong, Matthew Skalak, Howard Li, Kaitlin Allen, Jeff Hasty, and Sangeeta N Bhatia. Programmable probiotics for detection of cancer in urine. Science Translational Medicine, 7 (289): 289ra84-289ra84, 05 2015. doi: 10.1126/scitranslmed.aaa3519.
65. Jonathan W. Kotula, S. Jordan Kerns, Lev A. Shaket, Layla Siraj, James J. Collins, Jeffrey C. Way, and Pamela A. Silver. Programmable bacteria detect and record an environmental signal in the mammalian gut. Proceedings of the National Academy of Sciences, 111 (13): 4838-4843, 2014. doi: 10.1073/pnas. 1321321111.
66. Robert M. Cooper, Josephine A. Wright, Jia Q. Ng, Jarrad M. Goyne, Nobumi Suzuki, Young K. Lee, Mari Ichinose, Georgette Radford, Feargal J. Ryan, Shalni Kumar, Elaine M. Thomas, Laura Vrbanac, Rob Knight, Susan L. Woods, Daniel L. Worthley, and Jeff Hasty. Engineered bacteria detect tumor DNA. Science, 381 (6658): 682-686, 2023. doi: 10.1126/science.adf3974.
67. Yu-Yu Cheng, Zhengyi Chen, Xinyun Cao, Tyler D. Ross, Tanya G. Falbel, Briana M. Burton, and Ophelia S. Venturelli. Programming bacteria for multiplexed DNA detection. Nature Communications, 14 (1): 2001, 2023. doi: 10.1038/s41467-023-37582-x.
68. Xuefei Angelina Nou and Christopher A. Voigt. Sentinel cells programmed to respond to envi-ronmental DNA including human sequences. Nature Chemical Biology, 20 (2): 211-220, 2024. doi: 10.1038/s41589-023-01431-1.
69. Shamfin Hossain Kasfy, Farin Tasnova Hia, and Tofazzal Islam. Do CRISPR-based disease diagnosis methods qualify as point-of-care diagnostics for plant diseases? The Nucleus, 67 (1): 11-24, 2024. doi: 10.1007/s13237-024-00489-z.
70. Magdy Mahfouz. Revolutionizing Point-of-Care Diagnostics via CRISPR-Systems. ACS Synthetic Biol-ogy, 13 (2): 411-412, 2024. doi: 10.1021/acssynbio.3c00763.
71. Yupiao Xia, Ruotong Rao, Mengqiu Xiong, Bangshun He, Bingxin Zheng, Yanwei Jia, Ying Li, and Yun-huang Yang. CRISPR-Powered Strategies for Amplification-Free Diagnostics of Infectious Diseases. Analytical Chemistry, 96 (20): 8091-8108, 2024. doi: 10.1021/acs.analchem.3c04363.
72. Qi Zhang, Duo Ma, Fuqing Wu, Kylie Standage-Beier, Xingwen Chen, Kaiyue Wu, Alexander A. Green, and Xiao Wang. Predictable control of RNA lifetime using engineered degradation-tuning RNAs. Nature Chemical Biology, 17 (7): 828-836, 2021. doi: 10.1038/s41589-021-00816-4.
73. Jie Xu, Zhujun Liu, Zhen Zhang, and Tongbo Wu. Unlocking the Full Potential of Cas12a: Exploring the Effects of Substrate and Reaction Conditions on Trans-Cleavage Activity. Analytical Chemistry, 95 (28): 10664-10669, 2023. doi: 10.1021/acs.analchem.3c01307.
74. Diego A. Huyke, Ashwin Ramachandran, Vladimir I. Bashkirov, Efthalia K. Kotseroglou, Theofilos Kotseroglou, and Juan G. Santiago. Enzyme Kinetics and Detector Sensitivity Determine Limits of Detection of Amplification-Free CRISPR-Cas12 and CRISPR-Cas13 Diagnostics. Analytical Chemistry, 94 (27): 9826-9834, 2022. doi: 10.1021/acs.analchem.2c01670.
75. Kathryn T Elliott and Ellen L Neidle. Acinetobacter baylyi ADP1: Transforming the choice of model organism. IUBMB Life, 63 (12): 1075-1080, 10 2011. doi: 10.1002/iub.530.
76. Shannon M Soucy, Jinling Huang, and Johann Peter Gogarten. Horizontal gene transfer: building the web of life. Nature Reviews Genetics, 16 (8): 472-482, 07 2015. doi: 10.1038/nrg3962.
77. Robert M Cooper, Lev Tsimring, and Jeff Hasty. Inter-species population dynamics enhance mi-crobial horizontal gene transfer and spread of antibiotic resistance. eLife, 6:8053, 11 2017. doi: 10.7554/elife.25950.
78. Ronald Palmen, Ben Vosman, Pieter Buijsman, Cornelis K. D. Breek, and Klaas J. Hellingwerf. Physio-logical characterization of natural transformation in Acinetobacter calcoaceticus. Microbiology, 139 (2): 295-305, 1993. doi: 10.1099/00221287-139-2-295.
79. Robert M. Cooper and Jeff Hasty. One-Day Construction of Multiplex Arrays to Harness Natural CRISPR-Cas Systems. ACS Synthetic Biology, 9 (5): 1129-1137, 05 2020. doi: 10.1021/acssynbio.9b00489.
80. Robert M. Cooper and Jeff Hasty. Reference Module in Life Sciences. In Brenner's Encyclopedia of Genetics, 3rd Edition. Elsevier, 12 2022. doi: 10.1016/b978-O-12-822563-9.00088-3.
81. Marek Basler. Type VI secretion system: secretion by a contractile nanomachine. Philosophical Transac-tions of the Royal Society B: Biological Sciences, 370 (1679): 20150021, 2015. doi: 10.1098/rstb.2015.0021.
82. William P. J. Smith, Andrea Vettiger, Julius Winter, Till Ryser, Laurie E. Comstock, Marek Basler, and Kevin R. Foster. The evolution of the type VI secretion system as a disintegration weapon. PLOS Biology, 18 (5): e3000720, 2020. doi: 10.1371/journal.pbio.3000720.
83. Elisa T. Granato, William P. J. Smith, and Kevin R. Foster. Collective protection against the type VI secretion system in bacteria. The ISME Journal, 17 (7): 1052-1062, 2023. doi: 10.1038/s41396-023-01401-4.
84. Robin Patel, Christopher R Polage, Jennifer Dien Bard, Larissa May, Francesca M Lee, Valeria Fabre, Mary K Hayden, Sarah D B Doernberg, David A Haake, Barbara W Trautner, Larissa Grigoryan, Ephraim L Tsalik, and Kimberly E Hanson. Envisioning Future Urinary Tract Infection Diagnostics. Clinical Infectious Diseases, 74 (7): 1284-1292, 2021. doi: 10.1093/cid/ciab749.
85. Lin Lin, Raffaella Capozzoli, Alexia Ferrand, Miro Plum, Andrea Vettiger, and Marek Basler. Subcel-lular localization of Type VI secretion system assembly in response to cell-cell contact. The EMBO Journal, 41 (13): e108595, 2022. doi: 10.15252/embj.2021108595.
86. Ana-Maria S ̌imundic'. Measures of Diagnostic Accuracy: Basic Definitions. Journal of the International Federation of Clinical Chemistry and Laboratory Medicine, 19 (4), 1 2009.
87. Patrick M Bossuyt, Johannes B Reitsma, David E Bruns, Constantine A Gatsonis, Paul P Glasziou, Les M Irwig, Jeroen G Lijmer, David Moher, Drummond Rennie, Henrica C W de Vet, and Standards for Reporting of Diagnostic Accuracy. Towards Complete and Accurate Reporting of Studies of Diag-nostic Accuracy: The STARD Initiative. Annals of Internal Medicine, 138 (1): 40, 2003. doi: 10.7326/0003-4819-138-1-200301070-00010.
88. Deborah J Simpson, Lisa F Dawson, John C Fry, Hilary J Rogers, and Martin J Day. Influence of flanking homology and insert size on the transformation frequency of Acinetobacter baylyi BD413. Environmental Biosafety Research, 6 (1-2): 55-69, 09 2007. doi: 10.1051/ebr: 2007027.
89. Nils Hülter and Wilfried Wackernagel. Frequent integration of short homologous DNA tracks dur-ing Acinetobacter baylyi transformation and influence of transcription and RecJ and SbcCD DNases. Microbiology, 154 (12): 3676-3685, 12 2008. doi: 10.1099/mic.0.2008/021378-0.
90. Patrícia Apura, Susana Domingues, Sandra C Viegas, and Cecília M Arraiano. Reprogramming bacteria with RNA regulators. Biochemical Society Transactions, 47 (5): 1279-1289, 10 2019. doi: 10.1042/bst20190173.
91. Jongmin Kim, Yu Zhou, Paul D. Carlson, Mario Teichmann, Soma Chaudhary, Friedrich C. Simmel, Pamela A. Silver, James J. Collins, Julius B. Lucks, Peng Yin, and Alexander A. Green. De novo-designed translation-repressing riboregulators for multi-input cellular logic. Nature Chemical Biology, 15 (12): 1173-1182, 2019. doi: 10.1038/s41589-019-0388-1.
92. Alexandra M. Westbrook and Julius B. Lucks. Achieving large dynamic range control of gene expres-sion with a compact RNA transcription-translation regulator. Nucleic Acids Research, 45 (9): gkx215-, 2017. doi: 10.1093/nar/gkx215.
93. Sanne E Klompe, Phuc L H Vo, Tyler S Halpin-Healy, and Samuel H Sternberg. Transposon-encoded CRISPR-Cas systems direct RNA-guided DNA integration.

Nature, 571 (7764): 219-225, 07 2019. doi: 10.1038/s41586-019-1323-z.
94. Jonathan Strecker, Alim Ladha, Zachary Gardner, Jonathan L Schmid-Burgk, Kira S Makarova, Eu-gene V Koonin, and Feng Zhang. RNA-guided DNA insertion with CRISPR-associated transposases. Science, 365 (6448): 48-53, 07 2019. doi: 10.1126/science.aax9181.
95. Phuc Leo H. Vo, Carlotta Ronda, Sanne E. Klompe, Ethan E. Chen, Christopher Acree, Harris H. Wang, and Samuel H. Sternberg. CRISPR RNA-guided integrases for high-efficiency, multiplexed bacterial genome engineering. Nature Biotechnology, 39 (4): 480-489, 2021. doi: 10.1038/s41587-020-00745-y.
96. Chin-Wei Chang, Vy Anh Truong, Nam Ngoc Pham, and Yu-Chen Hu. RNA-guided genome engineering: paradigm shift towards transposons. Trends in Biotechnology, 2024. doi: 10.1016/j.tibtech.2024.02.006.
97. Shan-Chi Hsieh and Joseph E Peters. Natural and Engineered Guide RNA-directed Transposition with CRISPR-Associated Tn7-like Transposons. Annual Review of Biochemistry, 2024. doi: 10.1146/annurev-biochem-030122-041908.
98. Nicole A Becker, Tanya L Schwab, Karl J Clark, and L. James Maher. Bacterial gene control by DNA looping using engineered dimeric transcription activator like effector (TALE) proteins. Nucleic Acids Research, 46 (5): 2690-2696, 2018. doi: 10.1093/nar/gky047.
99. A Cournac and J Plumbridge. DNA Looping in Prokaryotes: Experimental and Theoretical Ap-proaches. Journal of Bacteriology, 195 (6): 1109-1119, 2013. doi: 10.1128/jb.02038-12.
100. Jose M G Vilar and Leonor Saiz. DNA looping in gene regulation: from the assembly of macromolecular complexes to the control of transcriptional noise. Current Opinion in Genetics & Development, 15 (2): 136-144, 2005. doi: 10.1016/j.gde.2005.02.005.
101. S. Oehler, M. Amouyal, P. Kolkhof, B. von Wilcken-Bergmann, and B. Müller-Hill. Quality and position of the three lac operators of E. coli define efficiency of repression. The EMBO Journal, 13 (14): 3348-3355, 1994. doi: 10.1002/j.1460-2075.1994.tb06637.x.
102. Nicole A. Becker, Alexander M. Greiner, Justin P. Peters, and L. James Maher. Bacterial promoter repression by DNA looping without protein-protein binding competition. Nucleic Acids Research, 42 (9): 5495-5504, 2014. doi: 10.1093/nar/gku180.
103. Michael T. Petassi, Shan-Chi Hsieh, and Joseph E. Peters. Guide RNA Categorization Enables Target Site Choice in Tn7-CRISPR-Cas Transposons. Cell, 183 (7): 1757-1771.e18, 2020. doi: 10.1016/j.cell.2020.11.005.
104. Andriy Didovyk, Bartłomiej Borek, Jeff Hasty, and Lev Tsimring. Orthogonal Modular Gene Repres-sion in Escherichia coli Using Engineered CRISPR/Cas9. ACS Synthetic Biology, 5 (1): 81-88, 01 2016. doi: 10.1021/acssynbio.5b00147.
105. Vivek K Mutalik, Lei Qi, Joao C Guimaraes, Julius B Lucks, and Adam P Arkin. Rationally designed families of orthogonal RNA regulators of translation. Nature Chemical Biology, 8 (5): 447-454, 2012. doi: 10.1038/nchembio.919.
106. Julius B Lucks, Lei Qi, Vivek K Mutalik, Denise Wang, and A P Arkin. Versatile RNA-sensing tran-scriptional regulators for engineering genetic networks. Proceedings of the National Academy of Sciences, 108 (21): 8617-8622, 05 2011. doi: 10.1073/pnas. 1015741108.
107. David Bikard, Wenyan Jiang, Poulami Samai, Ann Hochschild, Feng Zhang, and Luciano A. Mar-raffini. Programmable repression and activation of bacterial gene expression using an engineered CRISPR-Cas system. Nucleic Acids Research, 41 (15): 7429-7437, 2013. doi: 10.1093/nar/gkt520.
108. George D. Lampe, Rebeca T. King, Tyler S. Halpin-Healy, Sanne E. Klompe, Marcus I. Hogan, Phuc Leo H. Vo, Stephen Tang, Alejandro Chavez, and Samuel H. Sternberg. Targeted DNA integration in human cells without double-strand breaks using CRISPR-associated transposases. Nature Biotechnol-ogy, 42 (1): 87-98, 2024. doi: 10.1038/s41587-023-01748-1.
109. Ruchi A. Mehta and Jason D. Kahn. Designed hyperstable lac Repressor.DNA loop topologies suggest alternative loop geometries 1 1 Edited by R. Ebright. Journal of Molecular Biology, 294 (1): 67-77, 1999. doi: 10.1006/jmbi. 1999.3244.
110. David H Tse, Nicole A Becker, Robert T Young, Wilma K Olson, Justin P Peters, Tanya L Schwab, Karl J Clark, and L James Maher. Designed architectural proteins that tune DNA looping in bacteria. Nucleic Acids Research, 49 (18): 10382-10396, 2021. doi: 10.1093/nar/gkab759.
111. Bradley W Biggs, Stacy R Bedore, Erika Arvay, Shu Huang, Harshith Subramanian, Emily A McIntyre, Chantel V Duscent-Maitland, Ellen L Neidle, and Keith E J Tyo. Development of a genetic toolset for the highly engineerable and metabolically versatile Acinetobacter baylyi ADP1. Nucleic Acids Research, 48 (9): 5169-5182, 2020. doi: 10.1093/nar/gkaa167.
112. Andrea Vettiger and Marek Basler. Type VI Secretion System Substrates Are Transferred and Reused among Sister Cells. Cell, 167 (1): 99-110.e12, 09 2016. doi: 10.1016/j.cell.2016.08.023.
113. Larry C Anthony, Hideki Suzuki, and Marcin Filutowicz. Tightly regulated vectors for the cloning and expression of toxic genes. Journal of Microbiological Methods, 58 (2): 243-250, 2004. doi: 10.1016/j.mimet.2004.04.003.
114. Yusuke Kato. Extremely Low Leakage Expression Systems Using Dual Transcriptional-Translational Control for Toxic Protein Production. International Journal of Molecular Sciences, 21 (3): 705, 2020. doi: 10.3390/ijms21030705.
115. Joanne M L Ho, Corwin A Miller, Sydney E Parks, Jacob R Mattia, and Matthew R Bennett. A sup-pressor tRNA-mediated feedforward loop eliminates leaky gene expression in bacteria. Nucleic Acids Research, 49 (5): e25-e25, 2020. doi: 10.1093/nar/gkaa1179.
116. F. Veronica Greco, Amir Pandi, Tobias J. Erb, Claire S. Grierson, and Thomas E. Gorochowski. Har-nessing the central dogma for stringent multi-level control of gene expression. Nature Communications, 12 (1): 1738, 2021. doi: 10.1038/s41467-021-21995-7.
117. Pankaj Mehta, Sidhartha Goyal, and Ned S Wingreen. A quantitative comparison of sRNA-based and protein-based gene regulation. Molecular Systems Biology, 4 (1): 221, 01 2008. doi: 10.1038/msb.2008.58.
118. Maria J. Gonzalez-Moa, Bieke Van Dorst, Ole Lagatie, Ann Verheyen, Lieven Stuyver, and Marco A. Biamonte. Proof-of-Concept Rapid Diagnostic Test for Onchocerciasis: Exploring Peptide Biomarkers and the Use of Gold Nanoshells as Reporter Nanoparticles. ACS Infectious Diseases, 4 (6): 912-917, 2018. doi: 10.1021/acsinfecdis.8b00031 119. Federico M. Ruiz, Elena Santillana, Mercedes Spínola-Amilibia, Eva Torreira, Esther Culebras, and Antonio Romero. Crystal Structure of Hcp from Acinetobacter baumannii: A Component of the Type VI Secretion System. PLOS ONE, 10 (6): e0129691, 2015. doi: 10.1371/journal.pone.0129691.
120. Per Wessman, Denny Mahlin, Sultan Akhtar, Stefano Rubino, Klaus Leifer, Vadim Kessler, and Sebas-tian Ha°kansson. Impact of matrix properties on the survival of freeze-dried bacteria. Journal of the Science of Food and Agriculture, 91 (14): 2518-2528, 2011. doi: 10.1002/jsfa.4343.
121. Miriam Sand, Veronique de Berardinis, Ana Mingote, Helena Santos, Stephan Gö ttig, Volker Müller, and Beate Averhoff. Salt adaptation in Acinetobacter baylyi: identification and characterization of a secondary glycine betaine transporter. Archives of Microbiology, 193 (10): 723-730, 2011. doi: 10.1007/s00203-011-0713-x.
122. Anica Scholz, Julia Stahl, Veronique de Berardinis, Volker Müller, and Beate Averhoff. Regulation of choline oxidation in Acinetobacter baylyi. Environmental Microbiology Reports, 8 (2): 316-322, 2016. doi: 10.1111/1758-2229.12382.
123. Per Wessman, Sebastian Ha kansson, Klaus Leifer, and Stefano Rubino. Formulations for Freeze-drying of Bacteria and Their Influence on Cell Survival. Journal of Visualized Experiments, (78), 2013. doi: 10.3791/4058.
124. Hansjörg Götzke, Markus Kilisch, Markel Martínez-Carranza, Shama Sograte-Idrissi, Abirami Rajavel, Thomas Schlichthaerle, Niklas Engels, Ralf Jungmann, Paºl Stenmark, Felipe Opazo, and Steffen Frey. The ALFA-tag is a highly versatile tool for nanobody-based bioscience applications. Nature Communi-cations, 10 (1): 4403, 2019. doi: 10.1038/s41467-019-12301-7.
125. Lin Lin, Emmanuelle Lezan, Alexander Schmidt, and Marek Basler. Abundance of bacterial Type VI secretion system components measured by targeted proteomics. Nature Communications, 10 (1): 2584, 2019. doi: 10.1038/s41467-019-10466-9.
126. Sun-Young Kim, Jennifer K. Parker, Monica Gonzalez-Magaldi, Mady S. Telford, Daniel J. Leahy, and Bryan W. Davies. Export of Diverse and Bioactive Small Proteins through a Type I Secretion System. Applied and Environmental Microbiology, 89 (5): e00335-23, 2023. doi: 10.1128/aem.00335-23.
127. M. Omar Din, Tal Danino, Arthur Prindle, Matt Skalak, Jangir Selimkhanov, Kaitlin Allen, Ellixis Julio, Eta Atolia, Lev S. Tsimring, Sangeeta N. Bhatia, and Jeff Hasty. Synchronized cycles of bacterial lysis for in vivo delivery. Nature, 536 (7614): 81-85, 07 2016. doi: 10.1038/nature18930.
128. Sara Hooshangi, Stephan Thiberge, and Ron Weiss. Ultrasensitivity and noise propagation in a syn-thetic transcriptional cascade. Proceedings of the National Academy of Sciences, 102 (10): 3581-3586, 2005. doi: 10.1073/pnas.0408507102.
129. Daniela Pinto, Stefano Vecchione, Hao Wu, Marco Mauri, Thorsten Mascher, and Georg Fritz. Engi-neering orthogonal synthetic timer circuits based on extracytoplasmic function factors. Nucleic Acids Research, 46 (14): gky614-, 2018. doi: 10.1093/nar/gky614.
130. Jingjie Du, Mark Khemmani, Thomas Halverson, Adriana Ene, Roberto Limeira, Lana Tinawi, Baylie R. Hochstedler-Kramer, Melline Fontes Noronha, Catherine Putonti, and Alan J. Wolfe. Cat-aloging the phylogenetic diversity of human bladder bacterial isolates. Genome Biology, 25 (1): 75, 2024. doi: 10.1186/s13059-024-03216-8.
131. Ana Kolarevic, Denitsa Yancheva, Gordana Kocic, and Andrija Smelcerovic. Deoxyribonuclease in-hibitors. European Journal of Medicinal Chemistry, 88:101-111, 2014. doi: 10.1016/j.ejmech.2014.07.040.
132. G. Prigent, C. Perillaud, M. Amara, A. Coutard, C. Blanc, and B. Pangon. Actinobaculum schaalii: A truly emerging pathogen? New Microbes and New Infections, 11:8-16, 2016.
133. Yajun Song, Philippe Roumagnac, Franc,ois-Xavier Weill, John Wain, Christiane Dolecek, Camila J. Mazzoni, Kathryn E. Holt, and Mark Achtman. A multiplex single nucleotide polymorphism typing assay for detecting mutations that result in decreased fluoroquinolone susceptibility in Salmonella enterica serovars Typhi and Paratyphi A. Journal of Antimicrobial Chemotherapy, 65 (8): 1631-1641, 2010. doi: 10.1093/jac/dkq175.
134. Florence Hommais, Sabrina Pereira, Cécile Acquaviva, Patricia Escobar-Páramo, and Erick Denamur. Single-Nucleotide Polymorphism Phylotyping of Escherichia coli†. Applied and Environmental Microbi-ology, 71 (8): 4784-4792, 2005. doi: 10.1128/aem.71.8.4784-4792.2005.
135. Bertrand Picard, Jose Sevali Garcia, Stephanie Gouriou, Patrick Duriez, Naima Brahimi, Edouard Bin-gen, Jacques Elion, and Erick Denamur. The Link between Phylogeny and Virulence in Escherichia coli Extraintestinal Infection. Infection and Immunity, 67 (2): 546-553, 1999. doi: 10.1128/iai.67.2.546-553.1999.
136. Anna Sintsova, Arwen E Frick-Cheng, Sara Smith, Ali Pirani, Sargurunathan Subashchandrabose, Evan S Snitkin, and Harry Mobley. Genetically diverse uropathogenic Escherichia coli adopt a com-mon transcriptional program in patients with UTIs. eLife, 8: e49748, 2019. doi: 10.7554/elife.49748.
137. Seyedeh Elham Rezatofighi, Mahsa Mirzarazi, and Mansour Salehi. Virulence genes and phylo-genetic groups of uropathogenic Escherichia coli isolates from patients with urinary tract infection and uninfected control subjects: a case-control study. BMC Infectious Diseases, 21 (1): 361, 2021. doi: 10.1186/s12879-021-06036-4.
138. Hisham R. Ibrahim, Shinji Higashiguchi, Mamoru Koketsu, Lekh R. Juneja, Mujo Kim, Takehiko Ya-mamoto, Yasushi Sugimoto, and Takayoshi Aoki. Partially Unfolded Lysozyme at Neutral pH Agglu-tinates and Kills Gram-Negative and Gram-Positive Bacteria through Membrane Damage Mechanism. Journal of Agricultural and Food Chemistry, 44 (12): 3799-3806, 1996. doi: 10.1021/jf960133×.
139. D. Deckers, B. Masschalck, A. Aertsen, L. Callewaert, C. G. M. Van Tiggelen, M. Atanassova, and C. W. Michiels. Periplasmic lysozyme inhibitor contributes to lysozyme resistance in Escherichia coli. Cellular and Molecular Life Sciences CMLS, 61 (10): 1229-1237, 2004. doi: 10.1007/s00018-004-4066-3.
140. Chantal Abergel, Vincent Monchois, Deborah Byrne, Sabine Chenivesse, Frédérique Lembo, Jean-Claude Lazzaroni, and Jean-Michel Claverie. Structure and evolution of the Ivy protein family, un-expected lysozyme inhibitors in Gram-negative bacteria. Proceedings of the National Academy of Sciences, 104 (15): 6394-6399, 2007. doi: 10.1073/pnas.0611019104.
141. Colleen G Leong, Rebecca A Bloomfield, Caroline A Boyd, Amber J Dornbusch, Leah Lieber, Flora Liu, Amie Owen, Erin Slay, Kristine M Lang, and C Phoebe Lostroh. The role of core and accessory type IV pilus genes in natural transformation and twitching motility in the bacterium Acinetobacter baylyi. PLOS ONE, 12 (8): e0182139, 08 2017. doi: 10.1371/journal.pone.0182139.
142. Neslihan Sarigul, Filiz Korkmaz, and I'lhan Kurultak. A New Artificial Urine Protocol to Better Imitate Human Urine. Scientific Reports, 9 (1): 20159, 2019. doi: 10.1038/s41598-019-56693-4.
143. Joanna T. Zhang, Andrew Lezia, Philip Emmanuele, Muyao Wu, Connor A. Olson, Adam M. Feist, and Jeff Hasty. Host evolution improves genetic circuit function in complex growth environments. bioRxiv, page 2024.03.13.583595, 2024. doi: 10.1101/2024.03.13.583595.
144. Robert M. Cooper and Jeff Hasty. Rapid Assembly of Multiplex Natural CRISPR Arrays. In M. To-fazzal Islam and Kutubuddin Ali Molla, editors, CRISPR-Cas Methods: Volume 2, Springer Protocols 3-81. Springer Nature, 2021. doi: 10.1007/978-1-0716-1657-46.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of detecting a target nucleic acid in a sample, the method comprising:
a) administering to the sample an effective amount of a composition comprising a bacterium, wherein the bacterium comprises:
a competence system comprising a homologous recombination construct, wherein a first homology arm is substantially complementary to a sequence upstream of the target nucleic acid and a second homology arm is substantially complementary to a sequence downstream of the target nucleic acid,
an output gene construct encoding an output signal, and optionally,
a CRISPR/Cas system comprising an effector nuclease and a spacer directed to a non-targeted nucleic acid, and
b) detecting the output signal.

2. The method of claim 1, wherein the output signal is repressed by a repressor.

3. The method of claim 2, wherein the repressor is located between the two homology arms.

4. The method of claim 1, wherein the output signal is repressed by DNA looping with a repressor binding site located outside a CRISPR-targeted transposon that recognizes the target nucleic acid.

5. The method of claim 1 further comprising a nuclease inactivated CRISPR effector fused to a portion of a split enzyme, wherein the CRISPR effector recognizes the target nucleic acid in the sample to produce the output signal of an activated split enzyme.

6. The method of claim 1, wherein the sample is an environmental sample or a biological sample.

7. The method of claim 6, wherein the environmental sample comprises an air sample, a water sample, a dust sample, an agricultural product sample, a plant sample, a sewage sample, or a soil sample.

8. The method of claim 6, wherein the biological sample is from an animal.

9. The method of claim 1, wherein the competence system comprises a type IV pili.

10. The method of claim 1, wherein the target nucleic acid comprises a portion of an oncogene, a bacterial gene, or a viral gene.

11. The method of claim 1, wherein the CRISPR/Cas system comprises an I-A, I-B, I-C, I-D, I-E, I-F1, I-F2, I-F3, I-G, II-A, II-B, II-C1, II-C2, III-A, III-B-, III-C, III-D, III-E, III-F, IV-A, IV-B, IV-C, V-A, V-B1, V-B2, V-C, V-D, V-E, V-F1, V-F2, V-F3, V-G, V-U1, V-U2, V-U4, V-U5, VI-A, VI-B1, VI-B2, VI-C, or a VI-D CRISPR-Cas system.

12. The method of claim 1, wherein the bacterium comprises Acinetobacter baylyi, Bacillus subtilis, Streptococcus pneumoniae, Neisseria gonorrhoeae, Haemophilus influenzae, Vibrio natriegens, Vibrio cholera or Streptococcus sanguinis.

13. The method of claim 10, wherein the oncogene is Kirsten rat sarcoma virus (KRAS), Tumor protein p53 (TP53), Mothers against decapentaplegic homolog 4 (SMAD4), or V-Raf murine sarcoma viral oncogene homolog B1 (BRAF).

14. The method of claim 8, wherein the animal is a mammal having a cancer or an infectious disease.

15. The method of claim 14, wherein the cancer is colorectal cancer (CRC) or pancreatic cancer.

16. The method of claim 14, wherein the infectious disease is a microbial infection or an antimicrobial resistant infection.

17. The method of claim 15, wherein the cancer is characterized by a mutated KRAS gene, a mutated TP53, a mutated SMAD4, or a mutated BRAF gene.

18. The method of claim 17, wherein the mutated KRAS gene comprises a G12D, a G13, or a Q61 mutation.

19. The method of claim 17, wherein the mutated TP53 gene comprises a R175 or a R248 mutation.

20. The method of claim 17, wherein the mutated SMAD4 gene comprises a R361 or a R265 mutation.

21. The method of claim 17, wherein the mutated BRAF gene comprises a BRAFV600E mutation.

22. The method of claim 1, wherein the repressor is a polypeptide repressor, an RNA-based repressor, or a CRISPR Cas system.

23. The method of claim 22, wherein the polypeptide repressor comprises a tetracycline repressor (tetR),a lac repressor (lacI), a CI repressor, a PhIF repressor, a IcaRA repressor, an AmtR repressor, a BetI repressor, a SrpR repressor, an Orf2 repressor, a BM3R1 repressor, a ButR repressor, a PhIF repressor, an AmeR repressor, a QacR repressor, a LmrA repressor, a PsrA repressor, a HlyIIR repressor, a McbR repressor, a ScbR repressor, a TarA repressor, a LitR repressor, a HapR repressor, or a SmcR repressor.

24. The method of claim 22, wherein the RNA-based repressor comprises a small RNA-based repressor, toehold repressor, 3-way junction repressor, pT181 dual transcriptional/translational repressor.

25. The method of claim 22, wherein the CRISPR Cas system comprises CRISPR inactivation system.

26. The method of claim 1, wherein the output signal comprises an antibiotic resistance polypeptide, a fluorescent polypeptide, a bioluminescent polypeptide, a polypeptide that causes a color change, an ultrasound contrast agent polypeptide, a lateral flow assay, or an activated split enzyme.


SEQUENCE LISTING (10 sequences)

SEQ ID NO: 1 - DNA, 27 bp, synthetic construct
  source 1..27: mol_type = other DNA
ggtggagtatttgatagtgtattaacc

SEQ ID NO: 2 - DNA, 21 bp, synthetic construct
  source 1..21: mol_type = other DNA
agaatggtcctgcaccagtaa

SEQ ID NO: 3 - DNA, 23 bp, synthetic construct
  source 1..23: mol_type = other DNA
cagaacagcagtctggctattta

SEQ ID NO: 4 - DNA, 22 bp, synthetic construct
  source 1..22: mol_type = other DNA
actgcagacgtgtatcgtaatg

SEQ ID NO: 5 - DNA, 27 bp, synthetic construct
  source 1..27: mol_type = other DNA
agcgtcgatggaggagtttgtaaatga

SEQ ID NO: 6 - DNA, 23 bp, synthetic construct
  source 1..23: mol_type = other DNA
caaaatcggctccgtcgatacta

SEQ ID NO: 7 - DNA, 19 bp, synthetic construct
  source 1..19: mol_type = other DNA
tagcatcaccttcaccctc

SEQ ID NO: 8 - DNA, 20 bp, synthetic construct
  source 1..20: mol_type = other DNA
agagtttgatcatggctcag

SEQ ID NO: 9 - DNA, 20 bp, synthetic construct
  source 1..20: mol_type = other DNA
agagtttgatcctggctcag

SEQ ID NO: 10 - DNA, 20 bp, synthetic construct
  source 1..20: mol_type = other DNA
cggttaccttgttacgactt